(19) United States
(12) Patent Application Publication
(10) Pub. No.: US 2009/0203054 A1
(43) Pub. Date: Aug. 13, 2009

(54) HDL Cholesterol Sensor Using Selective Surfactant

(76) Inventors: Lindy Murphy, Yarnton (GB); Carla Burrows, Yarnton (GB); Simon Bayly, Yarnton (GB); Katherine Wilkinson, Yarnton (GB); James Harris, Yarnton (GB)

Correspondence Address: QUARLES & BRADY LLP, 411 E. WISCONSIN AVENUE, SUITE 2040, MILWAUKEE, WI, 53202-4497, US

(21) Appl. No.: 12/298,878

(22) PCT Filed: May 14, 2007
(86) PCT No.: PCT/GB07/01765

§ 371 Date: Oct. 28, 2008

(30) Foreign Application Priority Data
May 12, 2006 (GB) 0609494.0
Dec. 22, 2006 (GB) 0625817.2

(51) Int. Cl. C12Q 1/60

(52) U.S. Cl. 435/11

(57) ABSTRACT

A method for the determination of the amount of cholesterol in high density lipoproteins in a high density lipoprotein containing sample, said method comprising reacting the sample with a surfactant which selectively reacts with high density lipoproteins in the sample, said surfactant being selected from sucrose esters, and maltosides, and measuring the amount of cholesterol in the high density lipoproteins, for example using an electrochemical technique.

### FIELD OF THE INVENTION

The present invention relates to a method for determining the amount of cholesterol bound to high density lipoproteins (HDL cholesterol) in a high density lipoprotein—(HDL) containing sample. The invention also relates to a composition and a kit for use in such a method.

### BACKGROUND TO THE INVENTION

Many epidemiological investigations have demonstrated the strong and independent inverse association of high density lipoprotein (HDL), measured in terms of either its cholesterol or apo AI content, to risk of coronary artery disease (CAD). It is said that the risk of CAD increases 2-3% for every 10 mg/L decrease in HDL cholesterol. Thus, higher HDL cholesterol concentrations are considered protective. The measurement of HDL cholesterol in characterizing risk for CAD and managing treatment of dyslipidemia has therefore become increasingly common in clinical laboratories.

Initial laboratory methods for HDL cholesterol measurement, adapted from research techniques, required a manual separation step with precipitation reagents, followed by analysis of the cholesterol content, most often by an automated chemistry analyzer. Typical separation steps involved the reaction of a precipitation reagent with low density lipoproteins (LDL), very low density lipoproteins (VLDL) and chylomicrons (CM) in order to form an aggregate of these components. The aggregate was then removed from the reaction vessel, for example by centrifugation, leaving an HDL-containing sample ready for analysis. Separation of the precipitate was essential in order that the precipitate did not interfere with the UV/Vis or calorimetric analysis techniques used.

More recently, a number of techniques have been developed which do not require prior separation of the various lipoprotein fractions. These methods have the advantage that a measurement can typically be achieved in a single step, or at least without the need for precipitation to be carried out. Automation of the measurement is therefore possible. In one such approach, certain surfactants are used which break down the various lipoprotein fractions at different rates. For example, a surfactant might initially react more quickly with HDL, and reaction with LDL might occur more slowly. By measurement of the cholesterol content at a specified time after addition of the surfactant, the measurement has been found to have a greater dependency on the HDL cholesterol content than on the LDL cholesterol content.

This approach, however, has not generated the required accuracy and reliability in its results and the measurements made still retain some degree of dependency on the content of cholesterol in LDL, VLDL and CM. A new approach is therefore required which provides a simple and yet reliable and accurate method for the measurement of the HDL cholesterol content of body fluids such as blood and plasma. The measurement should also have a reduced dependency, or be entirely independent, of the content of cholesterol bound to LDL, VLDL and CM in the test sample. Further, preferred methods will not employ specialist equipment, or require trained technicians to carry out.

### SUMMARY OF THE INVENTION

The present invention provides a method for the determination of the amount of cholesterol in high density lipoproteins in a high density lipoprotein containing sample, said method comprising contacting the sample with (a) a surfactant which selectively breaks down high density lipoproteins, said surfactant being selected from sucrose esters and maltosides, and measuring the amount of cholesterol in the high density lipoproteins. The surfactant is preferably one which attenuates the reaction of low density lipoproteins.

The surfactants used in the present invention provide a very high selectivity for HDL over LDL, VLDL and CM. Whilst previous surfactants have been shown to react at different rates with HDL compared with other lipoproteins, the surfactants of the present invention react almost exclusively with HDL, and do not react, or substantially do not react, with other lipoproteins. It is believed that HDL in the sample is solubilised leaving HDL cholesterol available for reaction, whilst cholesterol bound to other lipoprotein fractions remains bound within the lipoprotein structure and is unavailable for reaction. The present invention is not, however, bound by this mode of action. An alternative theory is that the surfactants selectively suppress reaction of LDL, allowing only HDL to react. The surfactants of the invention are therefore those which selectively enable HDL cholesterol to react in a cholesterol assay, whilst LDL cholesterol is substantially unable to react. The subsequent measurement of the cholesterol content of the sample is thus reflective of the HDL-cholesterol content only, and is substantially independent of the amount of cholesterol contained within other lipoprotein fractions. The method of the present invention is therefore highly selective for HDL and provides an accurate and reliable test for HDL-cholesterol.

The method of the invention has the further advantage of improved simplicity compared with prior art tests. An HDL cholesterol measurement can be obtained by reacting a sample with a single reagent mixture and making a single measurement of the cholesterol content. Further, a result can be obtained in a very short period of time, typically within a minute or a few minutes of addition of the sample.

The measurement of HDL cholesterol is typically carried out by reacting the sample with a cholesterol ester hydrolysing reagent and either cholesterol oxidase or cholesterol dehydrogenase. The present invention accordingly also provides a reagent mixture for use in a method for the determination of the amount of cholesterol in high density lipoproteins in a high density lipoprotein containing sample, the reagent mixture comprising
(a) a surfactant as defined herein which selectively breaks down high density lipoproteins and which optionally attenuates the reaction of low density lipoproteins;
(b) a cholesterol ester hydrolysing reagent; and
(c) cholesterol oxidase or cholesterol dehydrogenase.

Also provided is a kit for the determination of the amount of cholesterol in high density lipoproteins in a high density lipoprotein containing sample, the kit comprising (a) a surfactant as defined herein which selectively breaks down high density lipoproteins and which optionally attenuates the reaction of low density lipoproteins, (b) a cholesterol ester hydrolysing reagent, and (c) cholesterol oxidase or cholesterol dehydrogenase, and means for measuring the amount of cholesterol which reacts with the cholesterol oxidase or cholesterol dehydrogenase. The kit is typically an electrochemical device wherein the means for measuring the amount of cholesterol which reacts with the cholesterol oxidase or cholesterol dehydrogenase comprises
an electrochemical cell having a working electrode, a reference or pseudo reference electrode and optionally a separate counter electrode;
a power supply for applying a potential across the cell; and
a measuring instrument for measuring the resulting electrochemical response.

The present invention also provides a method of operating the kit of the invention, said method comprising
(i) contacting (1) the reagents (a), (b) and (c) and (2) a high density lipoprotein containing sample, with each other and with the electrodes;
(ii) applying a potential across the electrochemical cell; and
(iii) electrochemically detecting the amount of product formed by measuring the resulting electrochemical response.

### BRIEF DESCRIPTION OF THE FIGURES

FIG. 1 depicts the measured current (nA) versus time (sec) for the results of cholesterol measurements carried out on (a) HDL-containing serum, (b) LDL-containing serum and (c) delipidated serum using sucrose monocaprate as the surfactant.

FIG. 2 depicts a device according to an embodiment of the invention.

FIGS. 3 to 9 depict the response gradients (nA/mM) versus time (sec) for sensors containing a series of Cymal surfactants. HDL response gradients are shown by the solid line; LDL response gradients are shown by the dotted line.

FIG. 10 shows the response gradients (nA/mM) versus time (sec) for sensors containing sucrose monocaprate either with or without LiCl. Graphs A-D are for sensors containing 5% SMC and either 0, 250, 500 or 750 mM LiCl. HDL and LDL gradients of response are shown with closed and open symbols respectively.

FIG. 11 depicts current Iox (nA) vs lipoprotein concentration (mM), at 238 sec for (a) no surfactant; (b) 40 mM sucrose monododecanoate (SMD); and (c) 100 mM sucrose monododecanoate (SMD). HDL is shown in solid values and LDL in dashed line/open circles

FIG. 12 depicts HDL vs LDL differentiation (%) versus time (see) for sensors with no surfactant, 40 mM SMD and 100mM SMD.

FIG. 13 depicts current Iox (nA) versus lipoprotein concentration (mM) at 238 sec. HDL is shown as solid values and LDL as dashed lines/open values for (a) no surfactant; (b) 60 mM sucrose monocaprate (SMC); and (c) 100 mM SMC.

FIG. 14 depicts HDL vs LDL differentiation (%) versus time (sec) for sensors with no surfactant, 60 mM SMC and 100 mM SMC.

FIG. 15 shows HDL vs LDL differentiation (%) vs time (sec) for sensors using 0%, 5%, 7.5% and 10% SMC.

FIG. 16 depicts current Iox (nA) versus lipoprotein concentration (mM) at 98 sec for HDL (solid) and LDL (dashed line, outline points) for sensors using (a) 0% SMC, (b) 5% SMC, (c) 7.5% SMC and (d) 10% SMC.

FIG. 17 shows HDL vs LDL differentiation (%) vs time (sec) for sensors using 0%, 0.5%, 1.0% and 1.5% SMD.

FIG. 18 depicts current Iox (nA) versus lipoprotein concentration (mM) at 98 sec for HDL (solid) and LDL (dashed line, outline points) for sensors using (a) 0% SMD, (b) 0.5% SMD, (c) 1.0% SMD and (d) 1.5% SMD.

### DETAILED DESCRIPTION OF THE INVENTION

The present invention provides a method of selectively determining the HDL-cholesterol content of a sample, wherein the sample may contain other lipoproteins which bind to cholesterol, as well as HDL. Selectivity is achieved by contacting the sample with a specific surfactant which is highly selective for HDL over LDL, VLDL and CM. Thus, the surfactant makes available for measurement the cholesterol and cholesterol esters bound to HDL, whilst those bound to LDL, VLDL and CM remain bound to the lipoprotein structure and substantially do not react in the later measurement of the cholesterol content.

It is known that cholesterol and cholesterol esters are largely carried in the blood in lipoprotein particles. It is a matter of some debate as to the precise mechanism by which enzymes and surfactants enable such cholesterols to be made available to be oxidized by dehydrogenases. Therefore, throughout this specification, it is understood that terms such as ‘breaks down’ or ‘made available’ or ‘impart reactivity’ all relate to the process by which an analytic response is obtained from cholesterol(s) in any sample. However, we do not wish to be bound by any particular theory as to the mode of action.

The surfactants employed in the present invention are those which selectively break down high density lipoproteins in a sample. This means that the surfactant reacts preferentially with HDL compared with LDL, VLDL and CM. The surfactants can alternatively be defined as those which selectively enable HDL cholesterol to react in a cholesterol assay, typically to react with a cholesterol ester hydrolysing reagent and cholesterol oxidase or dehydrogenase. In the context of the present invention, a surfactant which selectively breaks down HDL, or a surfactant which selectively enables HDL cholesterol to react in a cholesterol assay, is typically a surfactant having a differentiation between HDL and LDL of at least 50%, preferably at least 60%, at least 70%, at least 80% or most preferably at least 90%.

The differentiation between HDL and LDL can be determined according to the equation (i):

$$Differentiation ( \% ) = \frac{{G}_{HDL} - {G}_{LDL}}{{G}_{HDL}} \times 100 \qquad ( i )$$

wherein Gₓ is the gradient of the measured response to X (e.g. the measured current vs the known concentration of X). The measured response may be any measured value which relates (or corresponds) to the lipoprotein concentration, for example which is proportional to the lipoprotein concentration.

The skilled person can therefore easily determine whether any given surfactant is one which selectively breaks down HDL by using the chosen surfactant to measure the HDL cholesterol content of a sample of known HDL cholesterol content, and correspondingly measuring the LDL cholesterol content of a sample of known LDL cholesterol content using the same procedure. The differentiation value can be calculated from the results. The procedure for measuring the HDL or LDL cholesterol contents is typically that described in Example 1 below, using the chosen surfactant.

In the present invention, the concentration of HDL is typically measured electrochemically by determining the current generated at an electrode on electrochemical conversion of cholesterol to cholestenone. Since the current relates to the measured value of the HDL cholesterol content, the measured current is typically used to determine the gradient.

The differentiation should be measured using a time lapse between addition of reagents to the sample and measurement of the cholesterol content which is the same as the time lapse to be used during HDL cholesterol testing. Such time lapse is typically in order of 3 minutes or less, preferably 120 seconds or less, 90 seconds or less or 60 seconds or less. In the context of the invention, a selective surfactant is typically a surfactant having a differentiation between HDL and LDL of at least 50%, preferably at least 60%, at least 70%, at least 80% or most preferably at least 90%, when measuring the cholesterol contents using the procedure described in Example 1 and a time lapse between addition of reagents to the sample and measurement of 62 seconds.

In a preferred embodiment, the selective surfactants of the invention substantially do not break down LDL. In the cholesterol test of the invention therefore the surfactant preferably attenuates the reaction of LDL. The precise mechanisms of the action of the selective surfactants of the invention are not fully understood and we would not wish to be bound by any particular suggested mechanism.

It is believed that there is a kinetic separation of the cholesterol responses derived from the free cholesterol or the cholesterol esters present in either the HDL or the LDL, with HDL response preceding the LDL response, giving rise to differentiation to HDL.

The kinetic separation of response could be due to a number of reasons: the preferential action of the surfactant on HDL or to the attenuation of the LDL response, or both mechanisms may operate together.

It is known that HDL and LDL have different membrane proteins. HDL contains ApoA1 and LDL contains ApoB. The surfactant may have selective action in solubilising these proteins and disrupting the lipoprotein particle. It is also known that surfactant can be selectively incorporated into lipoprotein particles of different type, this ability being related to their hydrophilic-lipophilic balance (HLB). Incorporation of surfactant into a lipoprotein particle can cause it to swell in size and affect its reactivity.

It is also known that the core of the LDL particle has crystalline behaviour, below a transition temperature which is approximately at room temperature. In other words, the triglycerides and cholesterol ester in the core of the LDL particle are ordered and the LDL particles can be said to exhibit crystallinity. This is expected to affect the reactivity of the cholesterol in the core of the LDL particle. It is possible that the surfactant does not disrupt the order of the core of the LDL particles, i.e. it is difficult for the cholesterol ester in the core to emerge from the surface membrane layer for reaction with lipase.

The attenuation of the LDL response may be caused by the particular structure of the selective surfactants of the invention. The surfactants have a hydrophilic portion (sugar portion) and a hydrophobic portion (alkyl chain), and it is possible that the alkyl chain penetrates the membrane of the lipoprotein particle, while the sugar portion remains tethered on the outside. It is possible that incorporation of the surfactant in such a manner in the LDL particles results in coverage of the outer membrane with sugar portions, which prevent close approach and reaction with the enzymes. The incorporation of the surfactant in the HDL particles does not have this effect, and may result in rapid breakdown of the HDL particles into smaller micelles which can react readily with enzymes.

It is also possible that the surfactants of the invention could cause selective binding to the LDL particles, thereby reducing or totally eliminating their reactivity and the reaction of the cholesterol contained within the LDL particles with lipase/dehydrogenase.

It is even possible that the preferential action of the surfactant could be to activate the lipase/dehydrogenase enzymes towards HDL and/or to suppress the action of the enzymes on LDL.

The skilled person can determine whether the LDL response is attenuated by a chosen surfactant by comparing the gradient G_{LDL} (surfactant) (as defined above) obtained in the presence of the chosen surfactant to the gradient G_{LDL} (blank) obtained in the absence of a surfactant. The relationship

$$\frac{{G}_{LDL} ( surfactant )}{{G}_{LDL} ( blank )}$$

is less than 1 in the case that the surfactant attenuates the LDL reaction. Typically, the relationship

$$\frac{{G}_{LDL} ( surfactant )}{{G}_{LDL} ( blank )}$$

is less than 0.8, preferably less than 0.5 or less than 0.3.

The gradient G_{LDL} can be measured by any means for determining LDL concentration. Typically, in the present invention the gradient of current vs known concentration is used. The method for determining gradient as set out in Example 1 or 12 may be used.

Examples of sucrose esters for use as the surfactants of the invention include sucrose moieties in which one or more HO— groups is independently replaced with a group RCOO—, wherein R is typically an alkyl or alkenyl group which may be linear, branched or cyclic, having up to 18 carbon atoms. Examples of sucrose esters include compounds of formula (I):

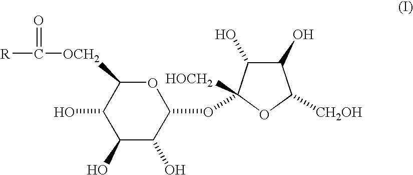

R is typically an alkyl or alkenyl group which may be linear, branched or cyclic, having up to 18 carbon atoms. Typically R is a linear alkyl group having at least 5, for example at least 7 carbon atoms. In one embodiment R has up to 15, for example up to 13 carbon atoms.

Further examples of sucrose esters are modifications of the compounds of formula (I) wherein the ester moiety appears at a different position on the sucrose moiety. Further examples include di- or poly-esters. In the case of di- or poly-esters, the two or more R groups may be the same or different, but are typically the same. Mixtures of two or more sucrose esters may be used.

Examples of maltosides for use as surfactants of the invention include those of formula (II):

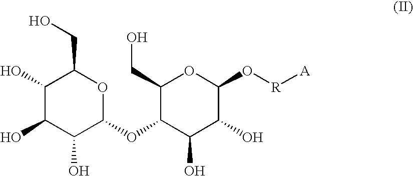

wherein R is an alkylene or alkenylene group having for example up to 18 carbon atoms and A is methyl or a cycloalkyl group having from 4 to 7 carbon atoms. R may be linear or branched. For example, R may be (CH₂)_{y}, wherein y is from 1 to 9.

In one embodiment, A is a cycloalkyl group having from 4 to 7 carbon atoms, preferably cyclohexyl. Examples of such compounds include cyclohexyl alkyl maltosides, such as cyclohexylmethyl-β-D-maltoside (Cymal-1, available from Anatrace), cyclohexylethyl-β-D-maltoside (Cymal-2, cyclohexylmethyl-β-D-maltoside, available from Anatrace), cyclohexylpropyl-β-D-maltoside (Cymal-3, available from Anatrace), cyclohexylbutyl-β-D-maltoside (Cymal-4, available from Anatrace), cyclohexylpentyl-β-D-maltoside (Cymal-5, available from Anatrace), cyclohexylhexyl-β-D-maltoside (Cymal-6, available from Anatrace) and cyclohexylheptyl-β-D-maltoside (Cymal-7, available from Anatrace).

In an alternative embodiment, A is methyl and R is an alkylene or alkenylene group having at least 5, e.g. at least 7 carbon atoms and having up to 15, e.g. up to 13 carbon atoms. Examples of such compounds include n-undecyl-β-D-maltoside, ω-undecylenyl-β-D-maltoside, n-octyl-β-D-maltoside, 2,6-dimethyl-4-heptyl-β-D-maltoside, 2-propyl-1-pentyl-β-D-maltoside, n-decyl-β-D-maltoside, n-tridecyl-β-D-maltoside, n-tetradecyl-β-D-maltoside and n-dodecyl-β-D-maltoside.

The above formula II depicts the β-maltosides. However, α-maltosides may also be employed as surfactants in the present invention. Further examples of surfactants therefore include the α equivalents of the maltosides listed above.

Preferred surfactants for use in the present invention include sucrose monocaprate, sucrose monodecanoate, Cymal-1, Cymal-2, Cymal-3, Cymal-4, Cymal-5, Cymal-6, Cymal-7, n-undecyl-α-D-maltoside, n-undecyl-β-D-maltoside, ω-undecylenyl-β-D-maltoside, n-octyl-β-D-maltoside, 2,6-dimethyl-4-heptyl-β-D-maltoside, 2-propyl-1-pentyl-β-D-maltoside, n-decyl-β-D-maltoside, n-tridecyl-β-D-maltoside, n-tetradecyl-β-D-maltoside and n-dodecyl-β-D-maltoside, in particular, sucrose monocaprate, sucrose monodecanoate, n-octyl-β-D-maltopyranoside, n-decyl-β-D-maltopyranoside, Cymal-4 and Cymal-5.

In one embodiment of the invention, preferred surfactants include, sucrose -monocaprate (Sigma Aldrich Co. Ltd), Cymal-1, Cymal-2, Cymal-3, Cymal-4, Cymal-5, Cymal-6 and Cymal-7. In another embodiment, preferred surfactants include sucrose monocaprate (Sigma Aldrich Co. Ltd), Cymal-4 and Cymal-5.

Surfactants may be used singly or two or more different surfactants may be used in combination. The total amount of surfactant used is typically up to 200 mg per ml of sample to be tested, preferably up to 100 mg/ml, for example about 50 mg/ml.

If desired, the sample may additionally be reacted with a complexing agent which forms a complex with lipoproteins other than HDL. Examples of complexing agents include polyanions, combinations of polyanions with divalent metal salts, and antibodies capable of binding to apoB containing lipoproteins. The polyanions may be selected from phosphotungstic acid and salts thereof, dextran sulphuric acid and salts thereof, polyethylene glycol and heparin and salts thereof. Once in complexed form, the lipoproteins other than HDL are unavailable for reaction and therefore do not interfere with the cholesterol measurement. However, due to the use of specific surfactants in the present invention, complexing agents are not required. It is accordingly preferred that the sample is not reacted with a complexing agent.

If desired, the sample may additionally be contacted with an ionic salt. The addition of an ionic salt has been found to result in faster kinetics of response to HDL. Suitable ionic salts include alkali metal (e.g. Li⁺, Na⁺, K⁺), alkaline earth metal (e.g. Mg²⁺, Ca²⁺) and transition metal (e.g. Cr³⁺) salts. LiCl, NaCl, MgCl₂, CaCl₂ and Cr(NH₃)₆Cl₃ are appropriate examples. In general, any ionic salt may be employed as long as it does not adversely affect the reaction, such as being oxidized or reduced under the measurement conditions used.

The measurement of the HDL-cholesterol content of the sample may be carried out by any suitable technique for measuring cholesterol. A preferred technique involves the reaction of the sample with a cholesterol ester hydrolysing reagent and cholesterol oxidase or cholesterol dehydrogenase. In one embodiment, cholesterol dehydrogenase is used, so the invention encompasses a method in which the sample is reacted with the surfactant and cholesterol dehydrogenase.

The cholesterol contained in HDL lipoproteins may be in the form of free cholesterol or cholesterol esters. A cholesterol ester hydrolysing reagent is therefore typically used to break down any cholesterol esters into free cholesterol. The free cholesterol is then reacted with the cholesterol oxidase or cholesterol dehydrogenase and the amount of cholesterol which has undergone such reaction is measured.

The cholesterol ester hydrolysing reagent may be any reagent capable of hydrolysing cholesterol esters to cholesterol. The reagent should be one which does not interfere with the reaction of cholesterol with cholesterol oxidase or cholesterol dehydrogenase and any subsequent steps in the assay. Preferred cholesterol ester hydrolysing reagents are enzymes, for example cholesterol esterase and lipases. Lipases are particularly preferred. A suitable lipase is, for example, a lipase from a pseudomonas or chromobacterium viscosum species. Commercially available enzymes, optionally containing additives such as stabilisers or preservatives may be used, e.g. those available from Toyobo or Amano. The cholesterol ester hydrolysing reagent may be used in an amount of from 0.1 to 25 mg per ml of sample, for example from 0.1 to 20 mg per ml of sample, preferably from 0.5 to 25 mg per ml, such as 0.5 to 15 mg per ml.

Any commercially available forms of cholesterol oxidase and cholesterol dehydrogenase may be employed. For instance, the cholesterol dehydrogenase is, for example, from the Nocardia species. The cholesterol oxidase or cholesterol dehydrogenase may be used in an amount of from 0.01 mg to 100 mg per ml of reagent mixture. In one embodiment, the cholesterol oxidase or dehydrogenase is used in an amount of from 0.1 to 20 mg per ml of sample, preferably from 0.5 to 25 mg per ml.

Each of the enzymes may contain additives such as stabilisers or preservatives. Further, each of the enzymes may be chemically modified.

The surfactant may be added to the sample prior to addition of the other reagents or simultaneously with the addition of the other reagents. In a preferred embodiment, the cholesterol ester hydrolysing reagent, cholesterol oxidase or dehydrogenase and surfactant are present in a single reagent mixture which is combined with the sample in a single step. In a particularly preferred embodiment, the method involves a single step of contacting the sample with reagents, so that only a single reagent mixture need be provided.

Measurements in accordance with the present invention can be carried out on any suitable sample containing HDL-cholesterol. Measurements are typically carried out on whole blood or blood components, for example serum or plasma. Preferred samples for use in the method of the present invention are serum and plasma. Where measurements are to be carried out on whole blood, the method may include the additional step of filtering the blood to remove red blood cells.

In a preferred embodiment of the invention, an electrochemical technique is used to measure the HDL-cholesterol content. This means that the amount of cholesterol which has reacted with the cholesterol oxidase or cholesterol dehydrogenase is determined by measuring an electrochemical response occurring at an electrode. In this embodiment, the sample is typically reacted with the surfactant, a cholesterol ester hydrolysing reagent, cholesterol oxidase or cholesterol dehydrogenase, a coenzyme capable of interacting with cholesterol oxidase or cholesterol dehydrogenase, and a redox agent which is capable of being oxidised or reduced to form a product which can be electrochemically detected at an electrode. The mixture of sample and reagents is contacted with a working electrode of an electrochemical cell so that redox reactions occurring can be detected. A potential is applied across the cell and the resulting electrochemical response, typically the current, is measured.

In this preferred embodiment, the amount of HDL-cholesterol is measured in accordance with the following assay:

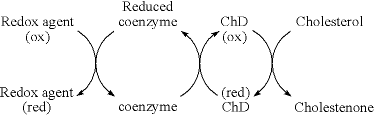

where ChD is cholesterol dehydrogenase. Cholesterol dehydrogenase could be replaced with cholesterol oxidase in this assay if desired. The amount of reduced redox agent produced by the assay is detected electrochemically. Additional reagents may also be included in this assay if appropriate.

Typically, the sample contacts all of the reagents in a single step. Therefore, a reagent mixture is provided which contains all of the required reagents and which can easily be contacted with the sample in order to carry out the assay. The reagent mixture of the invention typically comprises the surfactant in an amount of up to 200 mg, preferably up to 100 mg, for example about 50 mg per ml of sample, cholesterol ester hydrolysing reagent in an amount of from 0.1 to 20 mg, preferably from 0.5 to 20 mg per ml of sample and cholesterol dehydrogenase in an amount of from 0.1 to 30 mg, preferably from 0.5 to 25 mg per ml of sample.

Typically the coenzyme is NAD⁺ or an analogue thereof. An analogue of NAD⁺ is a compound having structural characteristics in common with NAD⁺ and which also acts as a coenzyme for cholesterol dehydrogenase. Examples of NAD⁺ analogues include APAD (Acetyl pyridine adenine dinucleotide); TNAD (Thio-NAD); AHD (acetyl pyridine hypoxanthine dinucleotide); NaAD (nicotinic acid adenine dinucleotide); NHD(nicotinamide hypoxanthine dinucleotide); and NGD (nicotinamide guanine dinucleotide). The coenzyme is typically present in the reagent mixture in an amount of from 1 to 20 mM, for example from 3 to 15 mM, preferably from 5 to 10 mM.

Typically, the redox agent should be one which can be reduced in accordance with the assay shown above. In this case, the redox agent should be one which is capable of accepting electrons from a coenzyme (or from a reductase as described below) and transferring the electrons to an electrode. The redox agent may be a molecule or an ionic complex. It may be a naturally occurring electron acceptor such as a protein or may be a synthetic molecule. The redox agent will typically have at least two oxidation states.

Preferably, the redox agent is an inorganic complex. The agent may comprise a metallic ion and will preferably have at least two valencies. In particular, the agent may comprise a transition metal ion and preferred transition metal ions include those of cobalt, copper, iron, chromium, manganese, nickel, osmium or ruthenium. The redox agent may be charged, for example it may be cationic or alternatively anionic. An example of a suitable cationic agent is a ruthenium complex such as Ru(NH₃)₆³⁺, an example of a suitable anionic agent is a ferricyanide complex such as Fe (CN)₆³⁻.

Examples of complexes which may be used include Cu(EDTA)²⁻, Fe(CN)₆³⁻, Fe(CN)₅(O₂CR)³⁻, Fe(CN)₄(oxalate)³⁻, Ru(NH₃)₆³⁺, Ru(acac)₂(Py-3-CO₂H)(Py-3-CO₂) and chelating amine ligand derivatives thereof (such as ethylenediamine), Ru(NH₃)₅(py)³⁺, ferrocenium and derivatives thereof with one or more of groups such as —NH₂, —NHR, —NHC(O)R, and —CO₂H substituted into one or both of the two cyclopentadienyl rings. Preferably the inorganic complex is Fe(CN)₆³⁻, Ru(NH₃)₆³⁺, Ru(acac)₂(Py-3-CO₂H)(Py-3-CO₂) or ferrocenium monocarboxylic acid (FMCA). Ru(NH₃)₆³⁺ or Ru(acac)₂(Py-3-CO₂H)(Py-3-CO₂) are preferred.

The redox agent is typically present in the reagent mixture in an amount of from 10 to 200 mM, for example from 20 to 150 mM, preferably from 30 to 100 mM, or up to 80 mM.

In a preferred embodiment, the reagent mixture used in the electrochemical assay additionally comprises a reductase. The reductase typically transfers two electrons from the reduced NAD and transfers two electrons to the redox agent. The use of a reductase therefore provides swift electron transfer.

Examples of reductases which can be used include diaphorase and cytochrome P450 reductases, in particular, the putidaredoxin reductase of the cytochrome P450_{cam} enzyme system from Pseudomonas putida, the flavin (FAD/FMN) domain of the P450_{BM-3} enzyme from Bacillus megaterium, spinach ferrodoxin reductase, rubredoxin reductase, adrenodoxin reductase, nitrate reductase, cytochrome b₅ reductase, corn nitrate reductase, terpredoxin reductase and yeast, rat, rabbit and human NADPH cytochrome P450 reductases. Preferred reductases for use in the present invention include diaphorase and putidaredoxin reductases.

The reductase may be a recombinant protein or a naturally occurring protein which has been purified or isolated. The reductase may have been mutated to improve its performance such as to optimise the speed at which it carries out the electron transfer or its substrate specificity.

The reductase is typically present in the reagent mixture in an amount of from 0.5 to 100 mg/ml, for example from 1 to 50 mg/ml, 1 to 30 mg/ml or from 2 to 20 mg/ml.

The reagents can optionally be dried, more preferably, the reagents can be freeze dried.

In a preferred embodiment of the invention, the general scheme of the electrochemical assay is as follows:

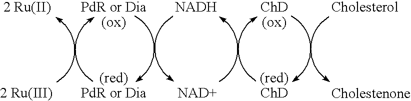

### Where

PdR—is putidaredoxin reductase
Dia—is diaphorase
ChD—is cholesterol dehydrogenase.

The reagent mixture optionally contains one or more additional components, for example excipients and/or buffers and/or stabilisers. Excipients are preferably included in the reagent mixture in order to stabilize the mixture and optionally, where the reagent mixture is dried onto the device of the invention, to provide porosity in the dried mixture. Examples of suitable excipients include sugars such as mannitol, inositol and lactose, and PEG. Glycine can also be used as an excipient. Buffers may also be included to provide the required pH for optimal enzyme activity. For example, a Tris buffer (pH9) may be used. Stabilisers may be added to enhance, for example, enzyme stability. Examples of suitable stabilisers are amino acids, e.g. glycine, and ectoine.

In a preferred embodiment, the reagent mixture for the electrochemical assay of the invention comprises a surfactant which selectively breaks down high density lipoproteins whilst showing attenuated action on LDL; cholesterol esterase or a lipase; cholesterol dehydrogenase; NAD⁺ or an analogue thereof; a reductase; and a redox agent. In a more preferred embodiment, the reagent mixture comprises a surfactant which selectively breaks down high density lipoproteins, cholesterol esterase or a lipase, cholesterol dehydrogenase, NAD⁺ or an analogue thereof, diaphorase or putidaredoxin reductase and Ru(NH₃)₆³⁺

The reagent mixture of the invention is typically provided in solid form, for example in dried form, or as gel. Alternatively it may be in the form of a solution or suspension. Whilst the amounts of each of the components present in the reaction mixture are expressed above in terms of molarity or w/v, the skilled person would be able to adapt these amounts to suitable units for a dried mixture or gel, so that the relative amounts of each component present remains the same.

Where an electrochemical measurement is carried out on whole blood, the measurement obtained may depend on the hematocrit. The measurement should therefore ideally be adjusted to at least partially account for this factor. Alternatively, the red blood cells can be removed by filtering the sample prior to carrying out the assay.

The present invention also provides a kit for selectively determining the HDL cholesterol content of an HDL-containing sample. The kit includes the required reagents, e.g. the surfactant, cholesterol ester hydrolysing reagent and cholesterol oxidase or cholesterol dehydrogenase, as well as means for measuring the amount of cholesterol which reacts with the oxidase or dehydrogenase.

In a preferred embodiment, the kit comprises a device for the electrochemical determination of the HDL-cholesterol content. In this embodiment, the means for determining the amount of cholesterol which has reacted includes an electrochemical cell having a working electrode, a reference electrode or pseudo reference electrode and optionally a separate counter electrode; a power supply for supplying a potential across the cell; and a measuring instrument for measuring the resulting electrochemical response, typically the current across the cell.

The device for electrochemical determination typically includes a reagent mixture as described above. The reagents may be present in the kit individually or in the form of one or more reagent mixtures. A single regent mixture is preferred. The reagent mixture may be present in the device in either liquid or solid form, but is preferably in solid form.

Typically, the reagent mixture is inserted into or placed onto the device whilst suspended/dissolved in a suitable liquid (e.g. water or buffer) and then dried in position. This step of drying the material into/onto the device helps to keep the material in the desired position. Drying may be carried out, for example, by air-drying, vacuum drying, freeze drying or oven drying (heating), preferably by freeze drying. The reagent mixture is typically located in the vicinity of the electrodes, such that when the sample contacts the reagent mixture, contact with the electrodes also occurs.

The device may optionally comprise a membrane through which the sample to be tested passes prior to contact with the reagent mixture. The membrane may, for example, be used to filter out components such as red blood cells, erythrocytes and/or lymphocytes. Suitable filtration membranes, including blood filtration membranes, are known in the art. Examples of blood filtration membranes are Presence 200 and PALL BTS SP300 of Pall filtration, Whatman VF2, Whatman Cyclopore, Spectral NX and Spectral X. Fibreglass filters, for example Whatman VF2, can separate plasma from whole blood and are suitable for use where a whole blood specimen is supplied to the device and the sample to be tested is plasma.

Alternative or additional membranes may also be used, including those which have undergone a hydrophilic or hydrophobic treatment prior to use. Other surface characteristics of the membrane may also be altered if desired. For example, treatments to modify the membrane's contact angle in water may be used in order to facilitate flow of the desired sample through the membrane. The membrane may comprise one, two or more layers of material, each of which may be the same or different. For example, conventional double layer membranes comprising two layers of different membrane materials may be used.

Appropriate devices for use in the present invention include those described in WO 2003/056319 and WO 2006/000828.

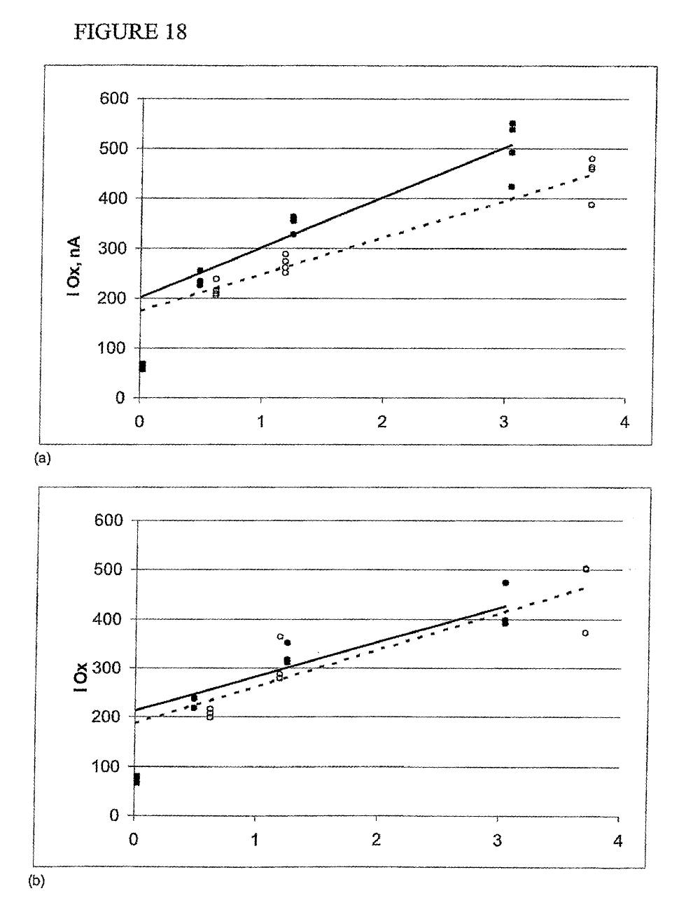

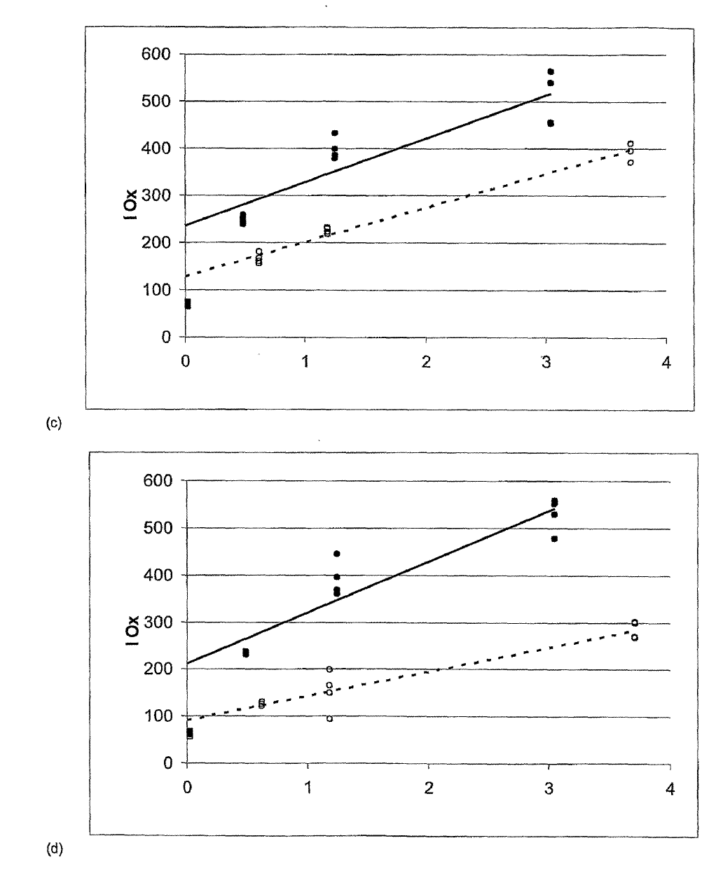

A device according to one embodiment of the invention is depicted in FIG. 18. In this embodiment, the working electrode 5 is a microelectrode. For the purposes of this invention, a microelectrode is an electrode having at least one working dimension not exceeding 50 μm.

The cell is in the form of a receptacle or a container having a base 1 and a wall or walls 2. Typically, the receptacle will have a depth (i.e. from top to base) of from 25 to 1000 μm. In one embodiment, the depth of the receptacle is from 50 to 500 μm, for example from 100 to 250 μm. In an alternative embodiment, the depth of the receptacle is from 50 to 1000 μm, preferably from 200 to 800 μm, for example from 300 to 600 μm. The length and width (i.e. from wall to wall), or in the case of a cylindrical receptacle the diameter, of the receptacle is typically from 0.1 to 5 mm, for example 0.5 to 1.5 mm, such as 1 mm.

The open end of the receptacle 3 may be partially covered by an impermeable material or covered by a semi-permeable or permeable material, such as a semi-permeable or permeable membrane. Preferably, the open end of the receptacle is substantially covered with a semi-permeable or permeable membrane 4. The membrane 4 serves, inter alia, to prevent dust or other contaminants from entering the receptacle.

The working electrode 5 is situated in a wall of the receptacle. The working electrode is, for example, in the form of a continuous band around the wall(s) of the receptacle. The thickness of the working electrode is typically from 0.01 to 25 μm, preferably from 0.05 to 15 μm, for example 0.1 to 20cm. Thicker working electrodes are also envisaged, for example electrodes having a thickness of from 0.1 to 50 μm, preferably from 5 to 20 μm. The thickness of the working electrode is its dimension in a vertical direction when the receptacle is placed on its base. The thickness of the working electrode is its effective working dimension, i.e. it is a dimension of the electrode which contacts the sample to be tested. The working electrode is preferably formed from carbon, palladium, gold or platinum, for example in the form of a conductive ink. The conductive ink may be a modified ink containing additional materials, for example platinum, graphite or platinised carbon. Two or more layers may be used to form the working electrode, the layers being formed of the same or different materials.

The cell also contains a pseudo reference electrode (not depicted) which may be present, for example, in the base of the receptacle, in a wall or walls of the receptacle or in an area of the device surrounding or close to the receptacle. The pseudo reference electrode is typically made from Ag/AgCl, although other materials may also be used. Suitable materials for use as the pseudo reference electrode will be known to the skilled person in the art. In this embodiment, the cell is a two-electrode system in which the pseudo reference electrode acts as both counter and reference electrodes. Alternative embodiments in which the cell comprises a reference electrode and a separate counter electrode can also be envisaged.

The pseudo reference (or reference) electrode typically has a surface area which is of a similar size to or smaller than, or which is larger than, for example substantially larger than, that of the working electrode 5. Typically, the ratio of the surface area of the pseudo reference (or reference) electrode to that of the working electrode is at least 1:1, for example at least 2:1 or at least 3:1. A preferred ratio is at least 4:1. The pseudo reference (or reference) electrode may, for example, be a macroelectrode. Preferred pseudo reference (or reference) electrodes have a dimension of 0.01 mm or greater, for example 0.1 mm or greater. This may be, for example, a diameter of 0.1 mm or greater. Typical areas of the pseudo reference (or reference) electrode are from 0.001 mm² to 100mm², preferably from 0.1 mm² to 60 mm², for example from 1 mm² to 50 mm². The minimum distance between the working electrode and the pseudo reference (or reference) electrode is, for example from 50 to 1000 μm.

In order that the cell can operate, the electrodes must each be separated by an insulating material 6. The insulating material is typically a polymer, for example, an acrylate, polyurethane, PET, polyolefin, polyester or any other stable insulating material. Polycarbonate and other plastics and ceramics are also suitable insulating materials. The insulating layer may be formed by solvent evaporation from a polymer solution. Liquids which harden after application may also be used, for example varnishes. Alternatively, cross-linkable polymer solutions may be used which are, for example, cross-linked by exposure to heat or UV or by mixing together the active parts of a two-component cross-linkable system. Dielectric inks may also be used to form insulating layers where appropriate. In an alternative embodiment, an insulating layer is laminated, for example thermally laminated, to the device.

The electrodes of the electrochemical cell may be connected to any required measuring instruments by any suitable means. Typically, the electrodes will be connected to electrically conducting tracks which are, or can be, themselves connected to the required measuring instruments.

The required reagents are typically contained within the receptacle, as depicted at 7 in FIG. 18. Typically, the reagents, in the form of a single reagent mixture, are inserted into the receptacle in liquid form and subsequently dried to help immobilise the composition. The reagent mixture, for example, may be air-dried, vacuum dried, freeze dried or oven-dried (heated), most preferably it is freeze dried.

The device of the present invention is operated by providing a sample to the device and enabling the sample to contact the reagent mixture. It is clear that sufficient time has to be allowed for plasma or blood to dissolve and react with the reagents in the mixture. Where plasma is used with freeze dried sensors, a time of approximately 20 s elapses between the application of the sample to the sensor and application of the applied potential of the cell. Where whole blood is used, this delay time may be longer to allow for blood cell removal, for example up to 5 minutes. In certain tests plasma is mixed with the reagents off the electrode and added to the cell with immediate application of potential. The potential is typically applied and the measurement read within a period of 10 seconds, typically 1-4 seconds.

The use of periods within this short range helps to ensure that the measurement detects only cholesterol bound to HDL. Where the surfactant used does react with LDL to a small extent, such reaction will be negligible and substantially will not affect the result where measurement is made within such short time periods. Typically, where Ru(II) is the product to be detected at the working electrode, the potential applied to the cell is from 0.1V to 0.3V. Preferred applied potential is 0.15V. (All voltages mentioned herein are quoted against a Ag/AgCl reference electrode, with 0.1M chloride). In a preferred embodiment, the potential is stepped first to a positive applied potential of 0.15V, for a period of about 1 second, and then stepped to a negative applied potential is then applied when it is desired to measure the reduction current. The use of the double potential step enables correction for electrode fouling and variation in electrode area to be minimized, as is described in WO 03/097860, incorporated herein by reference. Where a different redox agent is used, the applied potentials can be varied in accordance with the potentials at which the oxidation/reduction peak occurs.

The electrochemical test of the invention therefore enables a measurement of HDL cholesterol to be made in a very short period of time, typically within about 5 minutes, preferably within 3 minutes, 2 minutes or even 1 minute from application of a sample to the device. Results may in some circumstances be available in as little as 15 or 30 seconds from application of a sample to the device.

### EXAMPLES

### Example 1

### Preparation of Buffer Solution (Tris Buffer-5% Glycine pH9.0)

Trizma Pre-Set Crystals containing crystallised Tris and Tris HCl (pH 9.0, Sigma, T-1444) were dissolved in 950 mls dH₂O and the pH recorded. Following this 50g of Glycine (Sigma, G-7403) was added to the tris solution and the pH recorded. The pH was then adjusted to within 8.8-9.2 using 10M Potassium hydroxide (Sigma, P-5958) and the solution made up to 1000 mls with dH₂O and the final pH recorded (pH9.1). The solution was stored at 4° C. with an expiry date of 1 month.

### Preparation of Surfactant Solution

A surfactant solution was prepared by addition of sucrose monocaprate (Sigma) to the pre-prepared buffer solution to yield a 10% sucrose monocaprate buffered solution.

### Preparation of LDL & HDL Samples

LDL (Scipac, P232-8) and HDL (Scipac, P233-8) samples were made at 10× the required concentration (due to a 1:10 dilution in the final testing mixture) using dilipidated serum (Scipac, S139). The samples were then analysed using a Space clinical analyser (Schiappanelli Biosystems Inc). The approximate concentrations of LDL and HDL were 10 mM, giving 1 mM in the final testing mix.

### Preparation of Enzyme Mixture

An enzyme mixture was prepared by adding the following to the pre-prepared buffer solution:

### 160 mM Ruthenium Hexaammine (III) Chloride (Alfa Aesar, 10511)

17.7 mM Thionicotinamide adenine dinucleotide (Oriental Yeast Co)
8.4 mg/ml Putidaredoxin Reductase (Biocatalysts)
6.7 mg/ml Cholesterol Esterase (Sorachim/Toyobo, COE-311)
44.4 mg/ml Cholesterol Dehydrogenase, Gelatin free (Amano, CHDH-6)

### Testing Protocol

9 μl of the surfactant solution was mixed with 9 μl of the enzyme mixture. At T=−30 seconds, 2 μl of sample (either 10× concentrated LDL or 10× concentrated HDL, or 2 μl of delipidated serum) was mixed with the resulting surfactant:enzyme mix and 9 μl of the resulting solution placed onto an electrode. At T=0 seconds the chronoamperometry test was initiated. An oxidation potential of 0.15V is applied followed by a reduction potential of −0.45V. During application of the oxidation potential, the current is measured at 5 time points (T=0, 32, 64, 90 and 118 seconds). Each sample was tested in duplicate.

### Results

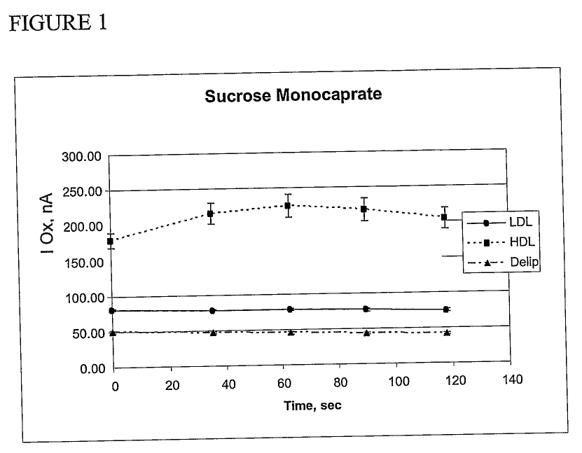

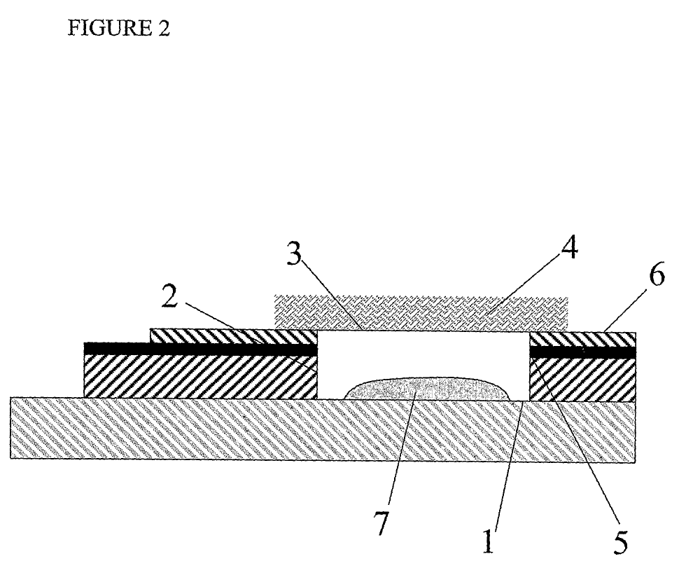

The results of the HDL and LDL test are depicted in FIG. 1, together with the delipidated sample as control. It is clear from FIG. 1 that sucrose monocaprate is selective for HDL over LDL, especially at short time periods.

In order to determine the differentiation value, the measured HDL current was plotted against known HDL concentration and the gradient at each time point was measured. Similarly, the gradient for each LDL measurement was calculated. The resulting differentiation values are shown in Table 1.

TABLE 1

| | Time (seconds) | | | | |
|---|---|---|---|---|---|
| | 0 | 32 | 63 | 90 | 118 |
| G_{LDL} (0.660) | 46.76 | 47.62 | 50.64 | 50.78 | 49.91 |
| G_{HDL} (0.932) | 139.88 | 180.69 | 193.18 | 188.57 | 175.59 |
| % Differentiation | 66.57 | 73.65 | 73.79 | 73.07 | 71.57 |

Where G_{LDL}(A) is the gradient of I_{LDL} VS known LDL cholesterol concentration at LDL cholesterol concentration A; and G_{HDL} (A) is the gradient of I_{HDL} vs known HDL cholesterol concentration at HDL cholesterol concentration A.

Sucrose monocaprate therefore demonstrates selectivity for HDL over LDL.

### Examples 2 to 8

### Preparation of Buffer Solution

The Buffer solutions contained 0.1M Tris (pH9.0), 30 mM KOH, 10% w/v β-Lactose. 30 mM mediator (mediator=Ru(acac)₂(Py-3-CO₂H)(Py-3-CO₂)) solution was made using 10% lactose buffer. This solution was mixed using a Covaris acoustic mixer using 1 cycle of 10 s with 50 cycles per burst with an intensity of 0.5 and 3 cycles of 60s with 100 bursts per cycle at an intensity of 5.

Enzymes and Cymal were added to the above buffer to the following final concentrations:

8.85 mM thionicotinamide adenine dinucleotide
4.2 mg/ml putidaredoxin reductase
3.35 mg/ml lipase (Genzyme, from Chromobacterium viscosum)
22.2 mg/ml cholesterol dehydrogenase, gelatin free
50 mM Cymal detergent

The Cymals used were:

Example 2: Cymal 1=2-cyclohexylmethyl-β-D-maltoside
Example 3: Cymal 2=2-cyclohexylethyl-β-D-maltoside

### Example 4: Cymal 3=Cyclohexylpropyl-β-D-maltoside

### Example 5: Cymal 4=Cyclohexylbutyl-β-D-maltoside

### Example 6: Cymal 5=Cyclohexylpentyl-β-D-maltoside

### Example 7: Cymal 6=Cyclohexylhexyl-β-D-maltoside

### Example 8: Cymal 7=Cyclohexylheptyl-β-D-maltoside

### Dispense and Freeze Drying

0.4 μl/well of each solution was dispensed onto sensors as described in WO 03056319. The dispensed sensor sheets were then freeze-dried.

### Samples

Testing was performed using previously frozen plasma samples. These were defrosted for a minimum of 30 minutes before being centrifuged at 14000 rpm for 5 minutes. Delipidated serum was also used as a sample. The samples were then analysed using a clinical analyser for TC, TG, HDL and LDL concentrations.

### Testing Protocol

Sensors were tested with plasma samples.

On the addition of 15 μl plasma sample to the sensor, the chronoamperometry test was performed using a multiplexer attached to an potentiostat This measured the oxidation current at +0.15 mV at 13 time points (0, 32, 64, 96, 128, 160, 192, 224, 256, 288, 320, 352 and 384 seconds), with a reduction current measured at −0.45 mV at the final time point (416 seconds). The transient was 4 seconds long and there was no delay time between each oxidation. Each plasma sample was tested in duplicate.

### Analysis

The results were analysed for the 4 second value on the current transients. The gradients at each time point were used to calculate the % differentiation obtained between measurement of LDL and HDL.

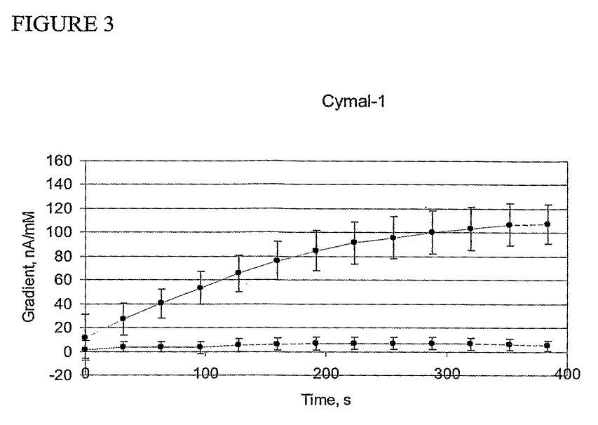

Results are shown in FIGS. 3 to 9, as follows:

FIG. 3: Response gradients (nA/mM) with time for sensors containing 50 mM Cymal-1. HDL response gradients are shown by the solid line, LDL response gradients are shown by the dotted line.

FIG. 4: Response gradients (nA/mM) with time for sensors containing 50 mM Cymal-2. HDL response gradients are shown by the solid line, LDL response gradients are shown by the dotted line.

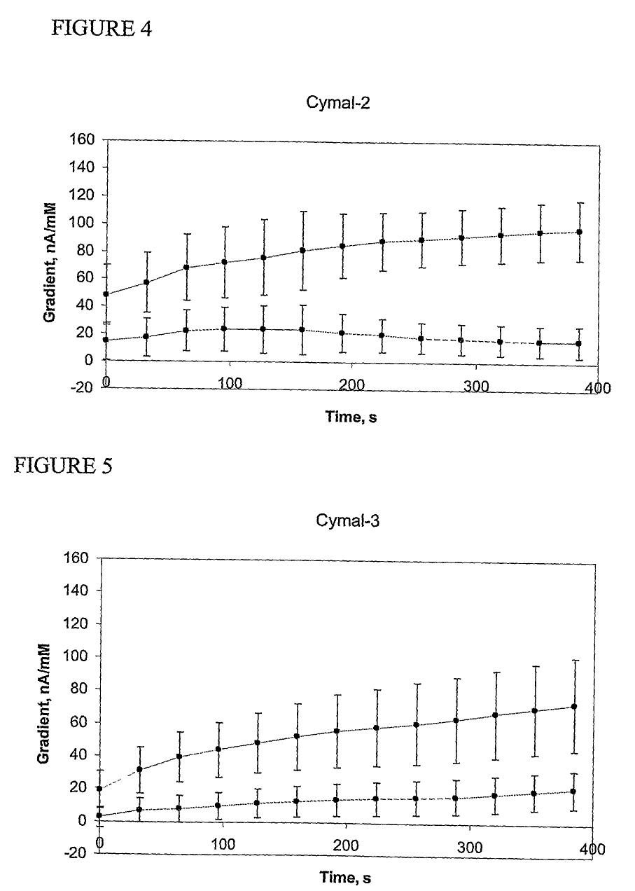

FIG. 5: Response gradients (nA/mM) with time for sensors containing 50 mM Cymal-3. HDL response gradients are shown by the solid line, LDL response gradients are shown by the dotted line.

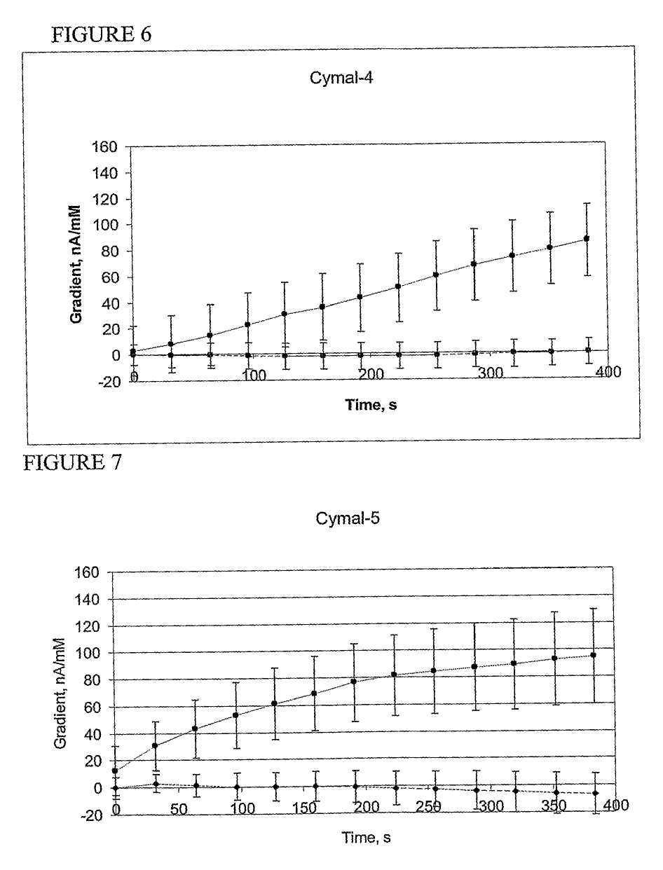

FIG. 6: Response gradients (nA/mM) with time for sensors containing 50 mM Cymal-4. HDL response gradients are shown by the solid line, LDL response gradients are shown by the dotted line.

FIG. 7: Response gradients (nA/mM) with time for sensors containing 50 mM Cymal-5. HDL response gradients are shown by the solid line, LDL response gradients are shown by the dotted line.

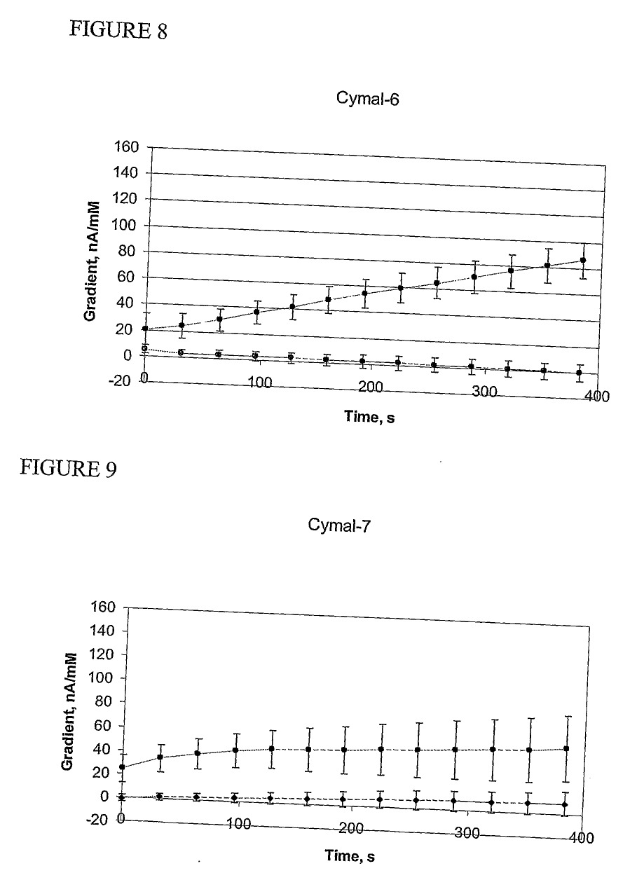

FIG. 8: Response gradients (nA/mM) with time for sensors containing 50 mM Cymal-6. HDL response gradients are shown by the solid line, LDL response gradients are shown by the dotted line.

FIG. 9: Response gradients (nA/mM) with time for sensors containing 50 mM Cymal-7. HDL response gradients are shown by the solid line, LDL response gradients are shown by the dotted line.

Comparison of the response to HDL and LDL for enzyme mixes containing Cymal surfactant shows that several Cymal surfactants show increased differentiation to HDL, most notably Cymals 4 and 5.

### Example 9

The aim of the experiment was to investigate the response to HDL of sensors containing either alpha or beta forms of n-undecyl-D-maltoside, or an alkyl maltoside with an unsaturated alkyl chain, ω-undecylenyl-β-D-maltoside.

### Preparation of Buffer Solution

The Buffer solutions contained 0.1M Tris (pH9.0), 30 mM KOH, 10% w/v β-Lactose. 30 mM mediator (mediator=Ru(acac)₂(Py-3-CO₂H)(Py-3-CO₂)) solution was made using 10% lactose buffer. This solution was mixed using a Covaris acoustic mixer using 1 cycle of 10 s with 50 cycles per burst with an intensity of 0.5 and 3 cycles of 60s with 100 bursts per cycle at an intensity of 5.

### Surfactant Solutions

Double strength maltoside solutions were made by adding maltoside to the pre-prepared RuAcac solution to produce the following final concentrations:

n-undecyl-α-D-maltopyranoside (Anatrace, U300HA)
200 mM (0.0179 g in 180 μl RuAcac solution)
100 mM (50 μl of 200 mM stock+50 μl RuAcac solution)
50 mM (25 μl of 200 mM stock+75 μl RuAcac solution)
n-undecyl-β-D-maltopyranoside (Sigma-Aldrich, 94206)
200 mM (0.0179 in 1801 RuAcac solution)
100 mM (50 μl of 200 mM stock+50 μl RuAcac solution)
50 mM (25 μl of 200 mM stock+75 μl RuAcac solution)
ω-undecylenyl-β-D-maltopyranoside (Anatrace, U310)
200 mM (0.0169 g in 171 μl RuAcac solution)
100 mM (50 μl of 200 mM stock+50 μl RuAcac solution)
50 mM (25 μl of 200 mM stock+75 μl RuAcac solution)

### Enzyme Mixture

Enzyme mixture was made at double strength by adding enzymes to the pre-prepared RuAcac solution to produce the following final concentrations:

17.7 mM Thionicotinamide adenine dinucleotide
8.4 mg/ml Putidaredoxin Reductase
6.7 mg/ml Lipase (Genzyme, from Chromobacterium viscosum)
44.4 mg/ml Cholesterol Dehydrogenase, Gelatin free

This solution was mixed using a Covaris acoustic mixer.

### Dispense and Freeze Drying

For each enzyme solution, equal volumes (approximately 50 ul) of double concentration enzyme solution and maltoside solution were mixed 1:1 to give the final enzyme/surfactant mixes. In addition, a blank mix was prepared by mixing equal volumes (approximately 50 ul) of double concentration enzyme solution and 30 mM RuAcac solution. 0.4 μl/well of each solution was dispensed onto sensors as described in WO 03056319 using an electronic pipette. The dispensed sensor sheets were then freeze dried.

### Samples

Testing was performed using previously frozen plasma samples. These were defrosted for a minimum of 30 minutes before being centrifuged at 1400 rpm. Delipidated serum (Scipac, S139) was also used as a sample. The samples were analysed using a Space clinical analyser (Schiappanelli Biosystems Inc) for TC, TG, HDL and LDL concentrations.

### Testing Protocol

Sensors were tested with plasma samples. On the addition of 12-15 ul plasma sample to the sensor, the chronoamperometry test was performed. This measured the oxidation current at +0.15 V at 13 time points (0, 32, 64, 96, 128, 160, 192, 224, 256, 288, 320, 352 and 384 seconds), with a reduction current measured at −0.45 V at the final time point (416 seconds). The transient was 4 seconds long and there was no delay time between each oxidation. Each plasma sample was tested in duplicate.

### Analysis

The output was analysed for the 4 second value on the current transients. The gradients of response to HDL and LDL at each time point were used to calculate the % differentiation obtained between measurement of LDL and HDL.

### Results

The gradients of response and % differentiation at 224 seconds are in the following table:

TABLE 2

| | HDL | LDL | |
|---|---|---|---|
| | gradient | gradient | |
| | at | at | |
| | 224 sec/ | 224 sec/ | % |
| sensor | nA/mM | nA/mM | differentiation |
| 100 mM | 34.6 | 14.7 | 57.6 |
| n-undecyl-α-D-maltopyranoside | | | |
| 50 mM | 52.8 | 14.6 | 72.3 |
| n-undecyl-α-D-maltopyranoside | | | |
| 25 mM | 61.4 | 7.1 | 88.5 |
| n-undecyl-α-D-maltopyranoside | | | |
| 100 mM | 22.4 | 14.6 | 34.8 |
| n-undecyl-β-D-maltopyranoside | | | |
| 50 mM | 37.7 | 14.6 | 61.4 |
| n-undecyl-β-D-maltopyranoside | | | |
| 25 mM | 32.8 | 11.2 | 66.0 |
| n-undecyl-β-D-maltopyranoside | | | |
| 100 mM | 32.1 | 14.2 | 55.8 |
| w-undecylenyl-β-D-maltopyranoside | | | |
| 50 mM | 54.3 | 11.2 | 79.4 |
| w-undecylenyl-β-D-maltopyranoside | | | |
| 25 mM | 44.0 | 9.4 | 78.6 |
| w-undecylenyl-β-D-maltopyroanside | | | |
| Blank | 23.4 | 21.4 | 8.4 |

Compared to the sensor responses with no added surfactant, the gradient of response and % differentiation to HDL are significantly increased by the use of any of these maltosides. It is concluded that both alpha and beta forms of alkyl maltosides can be effective differentiating agents in the HDL sensor. It is also concluded that alkyl maltosides with an unsaturated alkyl chain can be effective differentiating agents in the HDL sensor.

### Example 10

The aim of the experiment was to investigate the response to HDL of sensors containing alkyl-β-D-maltosides, with either linear or branched alkyl chains.

The method of Example 9 was repeated, but using the following as the double strength maltoside solutions:

n-octyl-β-D-maltopyranoside (Anatrace, 0310)
200 mM (0.0182 g in 200 μl RuAcac solution)
100 mM (50 μl of 200 mM stock+50 μl RuAcac solution)
50 mM (25 μl of 200 mM stock+75 μl RuAcac solution)
2,6-dimethyl-4-heptyl-β-D-maltopyranoside (Anatrace, DH325)
200 mM (0.0177 g in 189 μl RuAcac solution)
100 mM (50 μl of 200 mM stock+50 μl RuAcac solution)
50 mM (25 μl of 200 mM stock+75 μl RuAcac solution)
2-propyl-1-pentyl-β-D-maltopyranoside (Anatrace, P310)
200 mM (0.0175 g in 192 μl RuAcac solution)
100 mM (50 μl of 200 mM stock+50 μl RuAcac solution)
50 mM (25 μl of 200 mM stock+75 μl RuAcac solution)
n-decyl-β-D-maltopyranoside (Anatrace, D322)
200 mM (0.0184 g in 191 μl RuAcac solution)
100 mM (50 μl of 200 mM stock+50 μl RuAcac solution)
50 mM (25 μl of 200 mM stock+75 μl RuAcac solution)

### Results

The gradients of response and % differentiation at 224 seconds are in the following table:

TABLE 3

| | HDL | LDL | |
|---|---|---|---|
| | gradient | gradient | |
| | at | at | |
| | 224 sec/ | 224 sec/ | % |
| sensor | nA/mM | nA/mM | differentiation |
| 100 mM n-octyl-β-D-maltoside | 141.3 | 7.4 | 94.8 |
| 50 mM n-octyl-β-D-maltoside | 73.9 | 8.2 | 88.9 |
| 25 mM n-octyl-β-D-maltoside | 61.8 | 9.7 | 84.2 |
| 100 mM | 42.9 | 29.9 | 30.3 |
| 2,6-dimethyl-4-heptyl-β-D-maltoside | | | |
| 50 mM | 44.5 | 19.2 | 57.0 |
| 2,6-dimethyl-4-heptyl-β-D-maltoside | | | |
| 25 mM | 12.9 | 14.1 | −9.7 |
| 2,6-dimethyl-4-heptyl-β-D-maltoside | | | |
| 100 mM | 59.9 | 24.7 | 58.8 |
| 2-propyl-1-pentyl-β-D-maltoside | | | |
| 50 mM | 21.7 | 16.5 | 24.1 |
| 2-propyl-1-pentyl-β-D-maltoside | | | |
| 25 mM | 10.2 | 17.9 | −43.3 |
| 2-propyl-1-pentyl-β-D-maltoside | | | |
| 100 mM n-decyl-β-D-maltoside | 58.4 | 14.6 | 75.0 |
| 50 mM n-decyl-β-D-maltoside | 106.5 | 17.1 | 83.9 |
| 25 mM n-decyl-β-D-maltoside | 53.8 | 7.2 | 86.6 |
| Blank | 36.4 | 28.1 | 22.9 |

It is concluded that alkyl-β-D-maltosides with branched alkyl chains can act as differentiating agents in the HDL sensor.

### Example 11

The aim of the experiment was to investigate the response to HDL of sensors containing cymal-4 or cymal-5 (cyclohexyl-butyl-β-D-maltoside or cyclohexyl-pentyl-β-D-maltoside).

The method of Example 9 was repeated, but using the following as the double strength maltoside solutions:

### Cymal-4 (Anatrace, C324)

50 mM (0.0052 g in 217 μl RuAcac solution)

### Cymal-5 (Anatrace, C325)

50 mM (0.0051 g in 208 μl RuAcac solution)

### Results

The gradients of response and % differentiation at 224 seconds are in the following table:

TABLE 4

| | HDL gradient at | LDL gradient | |
|---|---|---|---|
| | 224 sec/ | at 224 sec/ | |
| sensor | nA/mM | nA/mM | % differentiation |
| blank | 14.5 | 21.6 | −32.7 |
| 25 mM cymal-4 | 43.9 | 6.1 | 86.2 |
| 25 mM cymal-5 | 47.5 | 5.6 | 88.1 |

It is concluded that both cymal-4 and cymal-5 can act as differentiating agents in the HDL sensor.

### Example 12

The aim of the experiment was to investigate the response to HDL of sensors containing n-alkyl-β-D-maltosides which have differing alkyl chain lengths.

RuAcac, maltoside and enzyme solutions were made up in accordance with Example 9, using the following double strength maltoside solutions:

n-octyl-β-D-maltopyranoside (Anatrace, 0310)
200 mM (0.0182 g in 201 μl RuAcac solution)
100 mM (50 μl of 200 mM stock+50 μl RuAcac solution)
50 mM (25 μl of 200 mM stock+75 μl RuAcac solution)
n-tridecyl-β-D-maltopyranoside (Anatrace, T323LA)
200 mM (0.0214 g in 204 μl RuAcac solution)
100 mM (50 μl of 200 mM stock+50 μl RuAcac solution)
50 mM (25 μl of 200 mM stock+75 μl RuAcac solution)
n-tetradecyl-β-D-maltopyranoside (Anatrace, T315)
200 mM (0.0215 g in 200 μl RuAcac solution)
100 mM (50 μl of 200 mM stock+50 μl RuAcac solution)
50 mM (25 μl of 200 mM stock+75 μl RuAcac solution)

### Dispense and Freeze Drying

The enzyme solutions were dispensed and freeze dried as described in Example 9. The samples were prepared in the same way as Example 9.

### Testing Protocol

12-15 μl of a plasma samples was used per electrode. On the addition of 12-15 μl of plasma the chronoamperometry test was initiated. The oxidation current is measured at 0.15 V at 13 time points (0, 32, 64, 96, 128, 160, 192, 224, 256, 288, 320, 352 and 384 seconds), with a reduction current measured at −0.45 V at the final time point (416 seconds). Each sample was tested in duplicate.

### Analysis

The gradients of response to HDL and LDL at each time point was used to calculate the % differentiation obtained between measurement of LDL and HDL.

### Results

The gradients of response and % differentiation at 224 seconds are in the following table:

TABLE 5

| | HDL | | |
|---|---|---|---|
| | gradient | LDL | |
| | at | gradient at | |
| | 224 sec/ | 224 sec/ | % |
| sensor | nA/mM | nA/mM | differentiation |
| 100 mM | 55.3 | 4.7 | 91.5 |
| n-octyl-β-D-maltopyranoside | | | |
| 50 mM | 76.7 | 6.1 | 92.0 |
| n-octyl-β-D-maltopyranoside | | | |
| 25 mM | 49.2 | 6.2 | 87.5 |
| n-octyl-β-D-maltopyranoside | | | |
| 100 mM | 38.2 | 13.4 | 64.9 |
| n-tridecyl-β-D-maltopyranoside | | | |
| 50 mM | 39.4 | 6.2 | 84.3 |
| n-tridecyl-β-D-maltopyranoside | | | |
| 25 mM | 26.9 | 4.4 | 83.7 |
| n-tridecyl-β-D-maltopyranoside | | | |
| 100 mM | 9.1 | 2.8 | 69.8 |
| n-tetradecyl-β-D-maltopyranoside | | | |
| 50 mM | 12.0 | 9.5 | 20.8 |
| n-tetradecyl-β-D-maltopyranoside | | | |
| 25 mM | 17.7 | 9.7 | 45.1 |
| n-tetradecyl-β-D-maltopyranoside | | | |
| Blank | 38.5 | 14.2 | 63.2 |

Compared to the sensor response with no added surfactant, the gradient of response and % differentiation to HDL are significantly increased by the use of n-octyl-β-D-maltoside. In addition, the gradient of response to LDL is reduced by the use of n-octyl-β-D-maltoside.

At 25 and 50 mM, n-tridecyl-β-D-maltoside did not increase the gradient of response to HDL but did decrease the gradient of response to LDL, and hence increased the % differentiation to HDL.

At 100 mM, n-tetradecyl-β-D-maltoside decreased the gradient of response to both HDL and LDL, and gave a small increase in the % differentiation to HDL.

### Example 13

The aim of the experiment was to investigate the response to HDL of sensors containing sucrose monocaprate (SMC) or n-octyl-β-D-maltoside (OMP), with either lipase or cholesterol esterase.

### Preparation of Buffer Solution

The buffer solution contained 0.1M Tris (pH9.0), 30 mM KOH, 10% w/v β-Lactose. 30 mM mediator (mediator=Ru(acac)₂(Py-3-CO₂H)(Py-3-CO₂)) solution was made using 10% lactose buffer. This solution was mixed using a Covaris acoustic mixer using 1 cycle of 10 s with 50 cycles per burst with an intensity of 0.5 and 3 cycles of 60 s with 100 bursts per cycle at an intensity of 5.

### Surfactant Solutions

Double strength surfactant solutions were made using RuAcac solution.

Sucrose monocaprate (SMC)(Dojindo, SO21-12)
200 mM (0.0217 g in 218 μl RuAcac solution)
n-octyl maltopyranoside (OMP) (Anatrace, 0310)
100 mM (0.0095 g in 209 μl RuAcac solution)

### Enzyme Mixture

Enzyme mixtures were made at double strength with either lipase (Genzyme, Chromobacterium viscosum) or cholesterol esterase (Genzyme, Pseudomonas sp.) by adding enzymes to the pre-prepared RuAcac solution to produce the following final concentrations:

17.7 mM Thionicotinamide adenine dinucleotide (Oriental Yeast Co)
8.4 mg/ml Putidaredoxin Reductase (Biocatalyst)
6.7 mg/ml Lipase (Genzyme) or cholesterol esterase (Genzyme)
44.4 mg/ml Cholesterol Dehydrogenase, Gelatin free (Amano, CHDH-6)

This solution was mixed using a Covaris acoustic mixer.

Further enzyme mixtures were made using the following lipases at quadruple strength:

Lipase from Chromobacterium viscosum (Genzyme, 70-1461-01)
0.0048 g in 89 uL of 100 mM SMC solution
Lipase from Pseudomonas sp. (Toyobo, LPL311)
0.0049 g in 91 uL of RuAcac solution.

These enzyme mixtures were diluted as appropriate by adding enzymes to the pre-prepared RuAcac solution to produce the following final concentrations:

8.8 mM Thionicotinamide adenine dinucleotide (Oriental Yeast Co)
4.2 mg/ml Putidaredoxin Reductase (Biocatalyst)
13.5 mg/ml Lipase (Genzyme) or lipase (Toyobo)
22.2 g/mL/ml Cholesterol Dehydrogenase, Gelatin free (Amano, CHDH-6)

### Final Enzyme Mixes

Equal volumes (approximately 50 ul) of double concentration enzyme solution and maltoside solution were mixed 1:1 to give the final single concentration enzyme/surfactant mixes. In addition, a blank mix was prepared by mixing equal volumes (approximately 50 ul) of double concentration enzyme solution and 30 mM RuAcac solution.

The quadruple concentration enzymes were diluted with RuAcac solution and surfactant solutions to give the final concentrations as shown above.

### Dispense and Freeze Drying

0.4 μl/well of each solution was dispensed onto sensors as described in WO 03056319 using an electronic pipette. The dispensed sensor sheets were then freeze dried.

### Samples

Testing was performed using previously frozen plasma samples. These were defrosted for a minimum of 30 minutes before being centrifuged at 1400 rpm for 5 minutes. Delipidated serum (Scipac, S139) was also used as a sample. The samples were analysed using a Space clinical analyser (Schiappanelli Biosystems Inc) for TC, TG, HDL and LDL concentrations.

### Testing Protocol

Sensors were tested with plasma samples. On the addition of 12-15 uL plasma sample to the sensor, the chronoamperometry test was performed. This measured the oxidation current at +0.15 V at 13 time points (0, 32, 64, 96, 128, 160, 192, 224, 256, 288, 320, 352 and 384 seconds), with a reduction current measured at −0.45 V at the final time point (416 seconds). The transient was 4 seconds long and there was no delay time between each oxidation. Each plasma sample was tested in duplicate.

### Analysis

The gradients of response to HDL and LDL at each time point were used to calculate the % differentiation obtained between measurement of LDL and HDL.

### Results

The gradients of response and % differentiation at 224 seconds are in the following table:

TABLE 6

| | HDL | | |
|---|---|---|---|
| | gradient | LDL | |
| | at | gradient at | |
| | 224 sec/ | 224 sec/ | % |
| sensor | nA/mM | nA/mM | differentiation |
| no surfactant, 1xGenzyme lipase | 14.5 | 21.6 | −32.7 |
| no surfactant, 1xGenzyme esterase | −3.8 | 19.7 | −119.0 |
| 100 mM sucrose monocaprate, | 37.6 | 1.5 | 96.0 |
| 1x Genzyme lipase | | | |
| 100 mM sucrose monocaprate, | 19.0 | −8.4 | 144.3 |
| 1x Genzyme esterase | | | |
| 50 mM n-octyl-β-D-maltoside, | 60.1 | 1.9 | 96.6 |
| 1x Genzyme lipase | | | |
| 50 mM n-octyl-β-D-maltoside, | 43.5 | 4.7 | 89.1 |
| 1x Genzyme esterase | | | |
| 100 mM sucrose monocaprate, | 72.8 | 9.2 | 87.4 |
| 4x Genzyme lipase | | | |
| 100 mM sucrose monocaprate, | 58.4 | 18.5 | 68.3 |
| 4x Toyobo lipase | | | |

Compared to the sensor responses with no added surfactant, the gradient of response and % differentiation to HDL are increased by the use of either lipase or cholesterol esterase with the surfactants SMC or OMP. The gradients of response to LDL are decreased for all mixes containing these surfactants compared to sensors with no added surfactant.

### Example 14

The aim of the experiment was to investigate the response to HDL of sensors containing n-alkyl-β-D-maltosides with different alkyl chain lengths.

The method of Example 12 was repeated, but using the following as the double strength maltoside solutions:

n-dodecyl-β-D-maltopyranoside (Anatrace, D310)
200 mM (0.0198 g in 194 μl RuAcac solution)
100 mM (50 μl of 200 mM stock+50 μl RuAcac solution)
50 mM (25 μl of 200 mM stock+75 μl RuAcac solution)
n-decyl-β-D-maltopyranoside (Anatrace, D322)
200 mM (0.0190 g in 197 μl RuAcac solution)
100 mM (50 μl of 200 mM stock+50 μl RuAcac solution)
50 mM (25 μl of 200 mM stock+75 μl RuAcac solution)
n-undecyl-β-D-maltopyranoside (Sigma-Aldrich, 94206)
200 mM (0.0173 g in 174 μl RuAcac solution)
100 mM (50 μl of 200 mM stock+50 μl RuAcac solution)
50 mM (25 μl of 200 mM stock+75 μl RuAcac solution)

### Results

The gradients of response and % differentiation at 224 seconds are in the following table:

TABLE 7

| | HDL | LDL | |
|---|---|---|---|
| | gradient at | gradient | |
| | 224 sec/ | at 224 sec/ | % |
| sensor | nA/mM | nA/mM | differentiation |
| No Surfactant | 39.5 | 14.0 | 64.7 |
| 100 mM | 46.7 | 7.4 | 84.3 |
| n-dodecyl-β-D-maltopyranoside | | | |
| 50 mM | 59.5 | 6.8 | 88.6 |
| n-dodecyl-β-D-maltopyranoside | | | |
| 25 mM | 58.9 | 7.8 | 86.7 |
| n-dodecyl-β-D-maltopyranoside | | | |
| 100 mM | 56.0 | 6.5 | 88.3 |
| n-decyl-β-D-maltopyranoside | | | |
| 50 mM | 56.6 | 6.4 | 88.8 |
| n-decyl-β-D-maltopyranoside | | | |
| 25 mM | 52.0 | 3.4 | 93.4 |
| n-decyl-β-D-maltopyranoside | | | |
| 100 mM | 34.4 | 6.6 | 80.7 |
| n-undecyl-β-D-maltopyranoside | | | |
| 50 mM | 50.6 | 6.3 | 87.5 |
| n-undecyl-β-D-maltopyranoside | | | |
| 25 mM | 75.4 | 7.1 | 90.6 |
| n-undecyl-β-D-maltopyranoside | | | |

Compared to the sensor response with no added surfactant, the gradient of response and % differentiation to HDL are significantly increased by the use of these n-alkyl-β-D-maltosides. In addition, the gradient of response to LDL is reduced by the use of these n-alkyl-β-D-maltosides.

### Example 15

The aim of the experiment was to investigate the response to HDL of sensors containing sucrose myristate or sucrose caprate. These surfactants contain a mix of mono and poly substituted sucrose esters. NMR analysis of these surfactants suggests they contain 1.1 alkyl chains per sucrose molecule.

The method of Example 12 was repeated, but using the following double strength surfactant solutions instead of the double strength maltoside solutions:

### Sucrose Myristate (Dojindo, S335)

10% (0.026 g in 260 μl RuAcac solution)
5% (50 μl of 10% stock+50 μl RuAcac solution)
2.5% (25 μl of 10% stock+75 μl RuAcac solution)

### Sucrose Caprate (Dojindo, S334)

10% (0.0248 g in 248 μl RuAcac solution)
5% (50 μl of 10% stock+50 μl RuAcac solution)
2.5% (25 μl of 10% stock+75 μl RuAcac solution)

### Results

The gradients of response and % differentiation at 224 seconds are in the following table:

TABLE 8

| | | LDL | |
|---|---|---|---|
| | HDL gradient | gradient at | |
| | at 224 sec/ | 224 sec/ | % |
| sensor | nA/mM | nA/mM | differentiation |
| 5% Sucrose Myristate | 56.0 | 12.6 | 77.5 |
| 2.5% Sucrose Myristate | 43.5 | 4.7 | 89.6 |
| 1.25% Sucrose Myristate | 50.6 | 6.7 | 86.8 |
| 5% Sucrose Caprate | 103.7 | 14.8 | 85.7 |
| 2.5% Sucrose Caprate | 100.8 | 3.9 | 96.1 |
| 1.25% Sucrose Caprate | 97.1 | 3.2 | 96.7 |
| Blank | 36.1 | 19.0 | 47.4 |

Compared to the sensor response with no added surfactant, the gradient of response and % differentiation to HDL are significantly increased by the use of sucrose caprate or sucrose myristate. In addition, the gradient of response to LDL is reduced by the use of sucrose caprate or sucrose myristate.

### Example 16

The aim of the experiment was to investigate the dependence of the response to HDL of sensors prepared with a wide range of concentrations of SMC or UMP.

The method of Example 12 was repeated, but using the following double strength surfactant solutions instead of the double strength maltoside solutions:

Sucrose monocaprate (Dojindo, SO21-12)
600 mM (0.0751 g in 252 μl RuAcac solution)
n-undecyl-β-D-maltopyranoside (Sigma-Aldrich, 94206)
600 mM (0.0749 g in 251 μl RuAcac solution)

| Dilutions | Volume 600 mM Stock | Volume RuAcac Solution |
|---|---|---|
| 400 mM | 60 μl | 30 μl |
| 200 mM | 30 μl | 60 μl |
| 100 mM | 15 μl | 75 μl |
| 50 mM | 10 μl | 110 μl |
| 20 mM | 3 μl | 87 μl |

### Results

The gradients of response and % differentiation at 224 seconds are in the following table:

TABLE 9

| | HDL | LDL | |
|---|---|---|---|
| | gradient | gradient at | |
| | at 224 sec/ | 224 sec/ | % |
| sensor | nA/mM | nA/mM | differentiation |
| 300 mM SMC | 55.6 | 0.0 | 100.0 |
| 200 mM SMC | 61.0 | 0.1 | 99.8 |
| 100 mM SMC | 98.2 | −1.5 | 101.6 |
| 50 mM SMC | 100.4 | −2.4 | 102.4 |
| 25 mM SMC | 108.8 | −2.8 | 102.5 |
| 10 mM SMC | 75.1 | 4.9 | 9.5 |
| 300 mM | 9.3 | 2.9 | 69.1 |
| undecyl-β-D-maltopyranoside | | | |
| 200 mM | 11.0 | 2.8 | 74.9 |
| undecyl-β-D-maltopyranoside | | | |
| 100 mM | 28.6 | 3.8 | 86.7 |
| undecyl-β-D-maltopyranoside | | | |
| 50 mM | 46.8 | 2.7 | 94.2 |
| undecyl-β-D-maltopyranoside | | | |
| 25 mM | 65.1 | −1.0 | 101.6 |
| undecyl-β-D-maltopyranoside | | | |
| 10 mM | 51.9 | 7.2 | 86.1 |
| undecyl-β-D-maltopyranoside | | | |
| Blank | 42.2 | 16.5 | 60.8 |

Compared to the sensor response with no added surfactant, the gradient of response and % differentiation to HDL are significantly increased by the use of SMC. In addition, the gradient of response to LDL is reduced by the use of SMC. The optimal amount of SMC is 25-100 mM, for highest HDL gradient of response.

Compared to the sensor response with no added surfactant, the gradient of response to HDL is increased at low concentrations of UMP and decreased at higher concentrations of UMP. In addition, the gradient of response to LDL is reduced by the use of UMP at all concentrations of UMP. Overall the % differentiation to HDL is significantly increased by the use of UMP at all concentrations. The optimal amount of UMP is 10-50 mM, for highest HDL gradient of response.

### Example 17

The aim of the experiment was to investigate the response to HDL with sensors prepared with 5% w/v SMC (sucrose monocaprate) and various ionic salts.

### Buffer Solutions

Buffer solution containing 10% β-Lactose (Sigma, L3750), 0.1M Tris PH9.0, 30 mM KOH was prepared. This was made up into a 5% w/v SMC (Dojindo SO21-12) solution by dissolution of 0.1 g SMC in 2.0 ml lactose buffer. Single strength RuAcac (Cis-[Ru(acac)₂(Py-3-CO₂H)(Py-3-CO₂)]) solution was made by dissolution of 0.0670g RuAcac in 4.083 ml of SMC solution. This solution was mixed using an acoustic mixer.

### Double Concentration Enzyme Solution

Enzyme solution was made at double concentration by adding enzymes to the pre-prepared 30 mM RuAcac solution. The enzyme solution contained the following:

17.7 mM Thionicotinamide adenine dinucleotide (Oriental Yeast Co)
8.4 mg/ml Putidaredoxin Reductase (Biocatalyst)
6.7 mg/ml Lipase (Genzyme, 1461)
44.4 mg/ml Cholesterol Dehydrogenase, Gelatin free (Amano, CHDH-6)

This solution was mixed using a Covaris acoustic mixer.

### Ionic Salt Solutions

Ionic salt solutions were prepared at double concentration by adding ionic salts to the pre-prepared RuAcac solution as follows:

### LiCl (Aldrich, 21, 323-3)

1.5 M LiCl solution: 0.0379 grams were dissolved in 595 μL of RuAcac solution.
1 M LiCl solution: 100 μL of 1.5 mM LiCl solution was mixed with 50 μl of RuAcac solution.
0.5 M LiCl solution: 50 μl of 1.5 mM LiCl solution was mixed with 100 μL of RuAcac solution.

### NaCl (Sigma, S-7653)

1 M NaCl solution: 0.0118 grams were dissolved in 201.7 μL of RuAcac solution.
100 mM NaCl solution: 1M NaCl solution and RuAcac solution were mixed in the ratio 1:9 by volume.

### MgCl₂ (Sigma, C-4901)

560 mM MgCl₂ solution: 0.0461 grams were dissolved in 403 μL of RuAcac solution.

### CaCl₂ (Sigma, M2670)

500 mM CaCl₂ solution: 0.0111 grams were dissolved in 200 μl of RuAcac solution.
250 mM CaCl₂ solution: 500 mM CaCl₂ solution and RuAcac solution were mixed in the ratio 1:1 by volume.

### Cr(NH₃)₆Cl₃ (Manchester Organics)

120 mM Cr(NH₃)₆Cl₃ solution: 0.0253 grams were dissolved in 809 μL of RuAcac solution.
30 mM Cr(NH₃)₆Cl₃: 37.5 μL of 120 mM Cr(NH₃)₆Cl₃ solution were mixed with 112.5 μL of RuAcac solution.

### Dispense and Freeze Drying

For each enzyme solution, equal volumes (approximately 50 ul) of double concentration enzyme solution and ionic salt solution were mixed 1:1 to give the final enzyme/ionic salt mixes. In addition, for each set of ionic salt experiments, a blank mix was prepared by mixing equal volumes (approximately 50 ul) each of double concentration enzyme solution and 30 mM RuAcac solution. 0.4 μl/well of each solution was dispensed onto sensors as described in WO 03056319 using an electronic pipette. The dispensed sensor sheets were then freeze dried.

### Samples

Testing was performed using previously frozen plasma samples. These were defrosted for a minimum of 30 minutes before being centrifuged at 1400 rpm for 5 minutes. Delipidated serum (Scipac, S139) was also used as a sample. The samples were analysed using a Space clinical analyser (Schiappanelli Biosystems Inc) for TC, TG, HDL and LDL concentrations.

### Testing Protocol

Sensors were tested with plasma samples. On the addition of 12-15 uL plasma sample to the sensor, the chronoamperometry test was performed. This measured the oxidation current at +0.15 V at 13 time points (0, 32, 64, 96, 128, 160, 192, 224, 256, 288, 320, 352 and 384 seconds), with a reduction current measured at −0.45 V at the final time point (416 seconds). The transient was 4 seconds long and there was no delay time between each oxidation. Each plasma sample was tested in duplicate.

### Analysis

The gradients of response to HDL and LDL at each time point were used to calculate the % differentiation obtained between measurement of LDL and HDL.

### Results

The use of ionic salt in the enzyme mix was found to decrease the amount of time taken for the sensor response to have maximum gradient of response to HDL. The time points at which of the sensor types gave maximum gradient of response to HDL are given in the table below:

TABLE 10

| | time at which HDL gradient reaches |
|---|---|
| chemistry | maxium value/seconds |
| 5% SMC blank | 384 |
| 5% SMC & 250 mM LiCl | 192 |
| 5% SMC & 500 mM LiCl | 160 |
| 5% SMC & 750 mM LiCl | 256 |
| 5% SMC blank | 352 |
| 5% SMC & 50 mM NaCl | 224 |
| 5% SMC & 500 mM NaCl | 160 |
| 5% SMC & 125 mM CaCl2 | 224 |
| 5% SMC & 250 mM CaCl2 | 288 |
| 5% SMC blank | 384 |
| 5% SMC & 280 mM MgCl2 | 256 |
| 5% SMC blank | 352 |
| 5% SMC & 15 mM Cr(NH3)6Cl3 | 192 |
| 5% SMC & 60 mM Cr(NH3)6Cl3 | 96 |

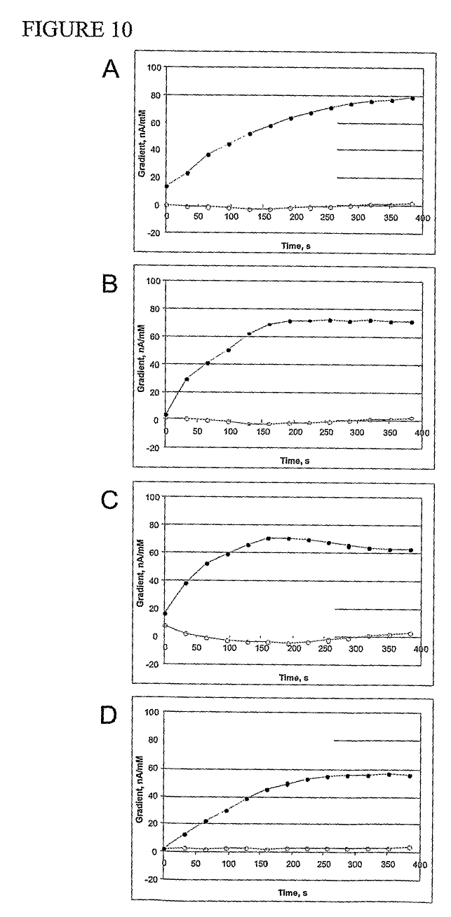

The gradients of response to HDL and LDL versus time are shown in FIG. 10 for sensors prepared with or without LiCl.

Addition of ionic salt generally resulted in faster kinetics of response to HDL, for HDL sensors prepared with 5% SMC.

### Example 18

The aim of the experiment was to investigate the response of sensors containing sucrose esters with different alkyl chain lengths to HDL.

### Preparation of Buffer Solution

The buffer solutions contained 0.1M Tris (pH9.0), 30 mM KOH, 10% w/v β-Lactose. 30 mM mediator (mediator=Ru(acac)₂(Py-3-CO₂H)(Py-3-CO₂)) solution was made using 10% lactose buffer. This solution was mixed using a Covaris acoustic mixer using 1 cycle of 10 s with 50 cycles per burst with an intensity of 0.5 and 3 cycles of 60 s with 100 bursts per cycle at an intensity of 5.

### Sucrose Ester Solutions

Double strength sucrose ester solutions were made by adding sucrose ester to the pre-prepared RuAcac solution to produce the following concentrations:

Sucrose monocaprate (SMC) (Dojindo, SO21-12)
200 mM (0.0201 g in 202 μl RuAcac solution)
100 mM (50 μl of 200 mM stock+50 μl RuAcac solution)
50 mM (25 μl of 200 mM stock+75 μl RuAcac solution)
n-Octanoylsucrose (SMO) (Calbiochem, 494466)
200 mM (0.0188 g in 201 μl RuAcac solution)
100 mM (50 μl of 200 mM stock+50 μl RuAcac solution)
50 mM (25 μl of 200 mM stock+75 μl RuAcac solution)
n-dodecanoylsucrose (SMD) (Calbiochem, 324374)
200 mM (0.0201 g in 202 μl RuAcac solution)
100 mM (50 μl of 200 mM stock+50 μl RuAcac solution)
50 mM (25 μl of 200 mM stock+75 μl RuAcac solution)

### Enzyme Mixture

Enzyme mixture was made at double strength by adding enzymes to the pre-prepared RuAcac solution to produce the following final concentrations:

17.7 mM Thionicotinamide adenine dinucleotide (Oriental Yeast Co)
8.4 mg/ml Putidaredoxin Reductase (Biocatalyst)
6.7 mg/ml Lipase (Genzyme, from Chromobacterium viscosum))
44.4 mg/ml Cholesterol Dehydrogenase, Gelatin free (Amano, CHDH-6)

This solution was mixed using a Covaris acoustic mixer, using Covaris S-series SonoLab-S1 software-Programme HDL 4° C.

### Dispense and Freeze Drying

For each enzyme solution, equal volumes (approximately 50 ul) of double concentration enzyme solution and sucrose ester solutions were mixed 1:1 to give the final enzyme/surfactant mixes. In addition, a blank mix was prepared by mixing equal volumes (approximately 50 ul) each of double concentration enzyme solution and 30 mM RuAcac solution. 0.4 μl/well of each solution was dispensed onto sensors as described in WO 03056319 using an electronic pipette. The dispensed sensor sheets were then freeze dried.

### Plasma Samples

Plasma samples were defrosted for 30 minutes before being centrifuged for 5 minutes at 2900 RCF. Delipidated serum (Scipac, S139) was also used as a sample. The samples were analysed using a Space clinical analyser (Schiappanelli Biosystems Inc) for TC, TG, HDL and LDL concentrations.

### Testing Protocol

12-15 μl of a plasma samples was used per electrode. On the addition of plasma the chronoamperometry test was initiated. The oxidation current was measured at 0.15 V at 13 time points (0, 32, 64, 96, 128, 160, 192, 224, 256, 288, 320, 352 and 384 seconds), with a reduction current measured at −0.45 V at the final time point (416 seconds). Each sample was tested in duplicate.

### Analysis

The gradients of response to HDL and LDL at each time point were used to calculate the % differentiation obtained between measurement of LDL and HDL.

### Results

The gradients of response and % differentiation at 224 seconds are in the following table:

TABLE 11

| | HDL gradient at | LDL gradient at | |
|---|---|---|---|
| | 224 sec/ | 224 sec/ | % |
| sensor | nA/mM | nA/mM | differentiation |
| No Surfactant | 29.6 | 15.8 | 46.7 |
| 100 mM SMC | 80.6 | 5.6 | 93.1 |
| 50 mM SMC | 71.7 | 1.5 | 97.4 |
| 25 mM SMC | 55.0 | −0.8 | 101.4 |
| 100 mM SMO | 98.4 | 8.6 | 91.3 |
| 50 mM SMO | 72.1 | 8.0 | 89.0 |
| 25 mM SMO | 72.4 | 9.6 | 86.7 |
| 100 mM SMD | 42.6 | 9.5 | 77.8 |
| 50 mM SMD | 93.1 | 12.0 | 87.1 |
| 25 mM SMD | 35.3 | 3.3 | 90.6 |

Compared to the sensor response with no added surfactant, the gradient of response and % differentiation to HDL are significantly increased by the use of sucrose ester. In addition, the gradient of response to LDL is reduced by the use of sucrose ester.

### Example 19

### Preparing Solutions, Dispensing and Freeze Drying

Several solutions were prepared using the same basic enzyme mix but different surfactants such that the final concentrations of components were:

### 0.1M Tris-HCL, pH 9.0

### 30 mM KCl

100 mg/ml Lactose
Surfactant (no surfactant (0 mg/ml), 40 mM Sucrose Monododecanoate (SMD)
(20 mg/ml), 100 mM SMD (50 mg/ml), 60 mM Sucrose Monocaprate (SMC)
(30 mg/ml) or 100 mM SMC (50 mg/ml)
30 mM Ruthenium AcAc mediator (Cis-[Ru(acac)₂(Py-3-CO₂H)(Py-3-CO₂)])
6 mg/ml Thio-nicotinamide adenine dinucleotide (TNAD)
4 mg/ml Putidaredoxin Reductase (PdR)
3 mg/ml Genzyme lipase (G. Lip)
22.2 mg/ml Cholesterol Dehydrogenase

0.4 ul of solution was dispensed per well onto a sensor as described in WO 0356319.

The sensors were freeze dried.

### Testing Procedure

20 ul of plasma was added to each electrode. At t=0 seconds the chronoamperometry test was initiated. The oxidation current was measured at +0.15V for 4 second at 34 second consecutive time intervals, for a period of 340 seconds, followed by a reduction current at −0.45V for 4 second. There was a 34 second delay between oxidations which resulted in oxidations at approximately 0, 34, 68, 102, 136, 170, 204, 238, 272, 306 and 340 seconds. Data was analysed for current values at 4 second on the transient.

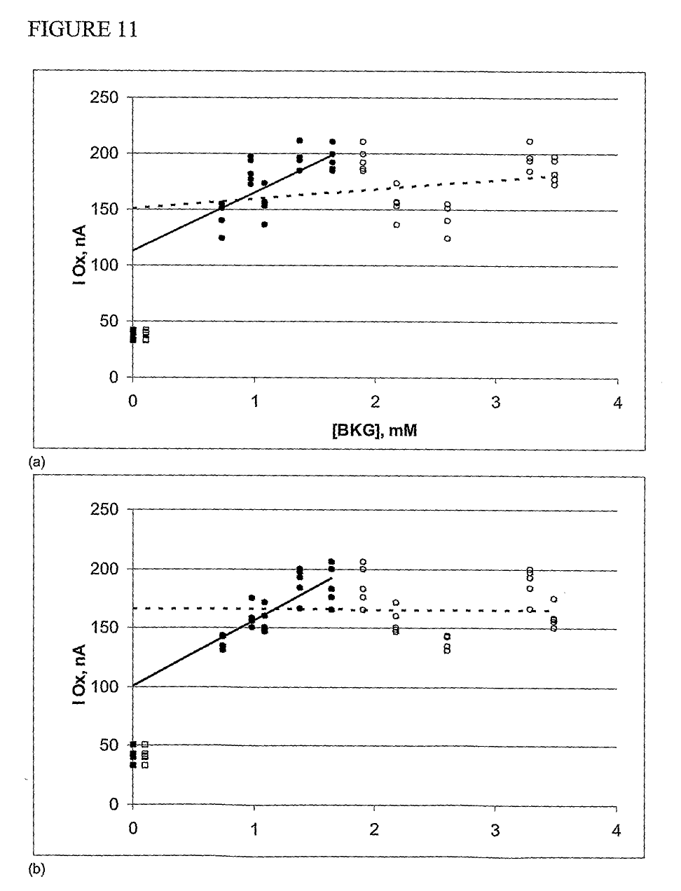

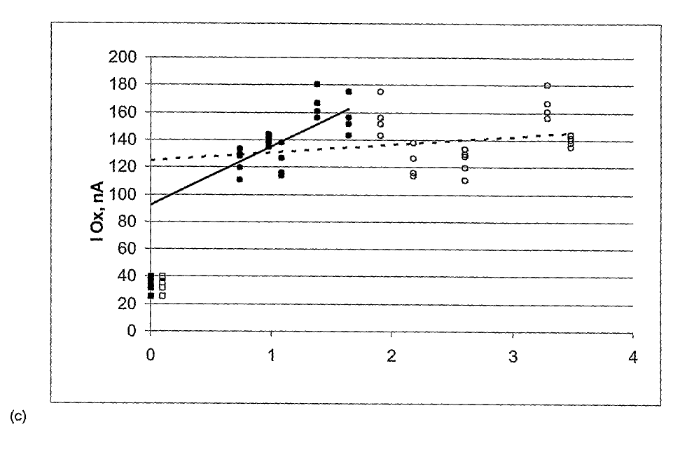

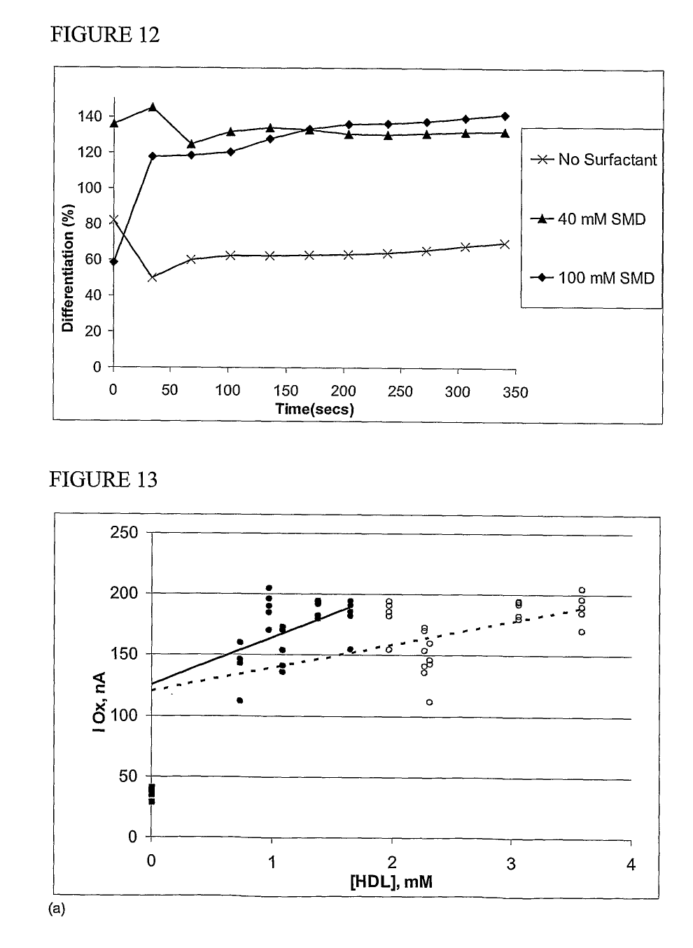

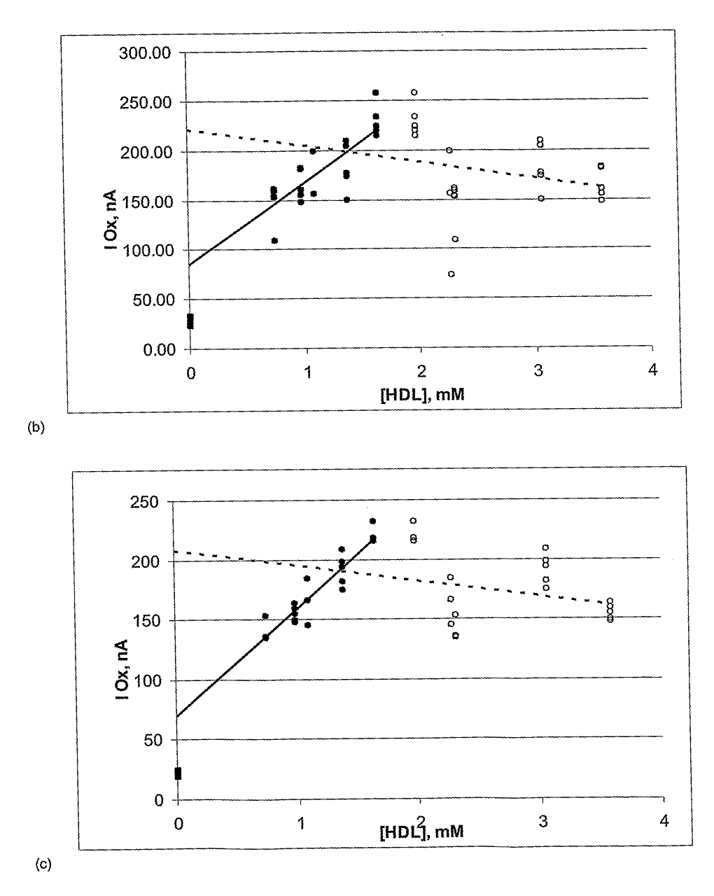

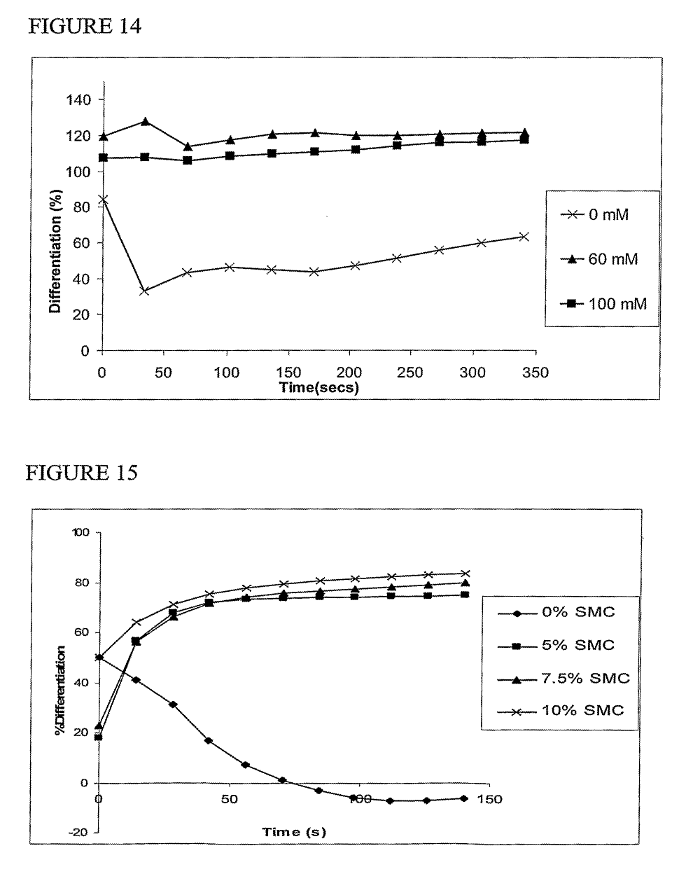

Results are depicted in FIGS. 11 to 14 and Tables 12 and 13:

TABLE 12
Differentiation with different concentrations of SMD at 238 seconds

| | Surfactant | | |
|---|---|---|---|
| | concentration | | |
| | No | 40 mM | 100 mM |
| | surfactant | SMD | SMD |
| HDL Gradient | 52.5 | 55.7 | 42.9 |
| (mM/nA) | | | |
| LDL Gradient | 19.0 | −16.6 | −15.6 |
| (mM/nA) | | | |
| Differentiation (%) | 63.8 | 129.9 | 136.3 |

TABLE 13
Differentiation with different concentrations of SMC at 238 seconds

| | Surfactant | | |
|---|---|---|---|
| | concentration | | |
| | No | 60 mM | 100 mM |
| | surfactant | SMC | SMC |
| HDL Gradient | 39.1 | 82.7 | 89.9 |
| (mM/nA) | | | |
| LDL Gradient | 19 | −16.6 | −12.9 |
| (mM/nA) | | | |
| Differentiation | 51.3 | 120.1 | 114.3 |
| (%) | | | |

### Example 20

### Solution Preparation, Dispense and Freeze Drying

Several solutions were prepared using the same basic enzyme mix but different surfactants such that the final concentrations of components were:

### 0.1M Tris-HCL, pH 9.0

50 mg/ml (5%) Glycine
Surfactant (0% SMC or no surfactant (0 mg/ml), 5% Sucrose Monocaprate (SMC) (50 mg/ml), 7.5% SMC (75 mg/ml) or 10% (100 mg/ml))
80 mM Ruthenium Hexaamine chloride
6 mg/ml Thio-nicotinamide adenine dinucleotide (TNAD)
4 mg/ml Putidaredoxin Reductase (PdR)
3 mg/ml Genzyme lipase
22.2 mg/ml Cholesterol Dehydrogenase

0.4 ul of solution was dispensed per well onto a sensor as described in WO 200356319. The sensors were freeze dried.

### Testing Procedure

20 ul of Scipac HDL or LDL prepared in delipidated serum was added to each electrode. The electrodes were tested by chronoamperometry using an Autolab and a multiplexer. At t=0 seconds the chronoamperometry tested. The oxidation current was measured at +0.15V for 1 second at 14 second consecutive time intervals, for a period of 140 seconds, followed by a reduction current at −0.45V for 1 second. There was a 14 second delay between oxidations which resulted in oxidations at approximately 0, 14, 28, 42, 56, 70, 84, 98, 112, 126 and 140 seconds. Data was analysed for current values at 1 second on the transient.

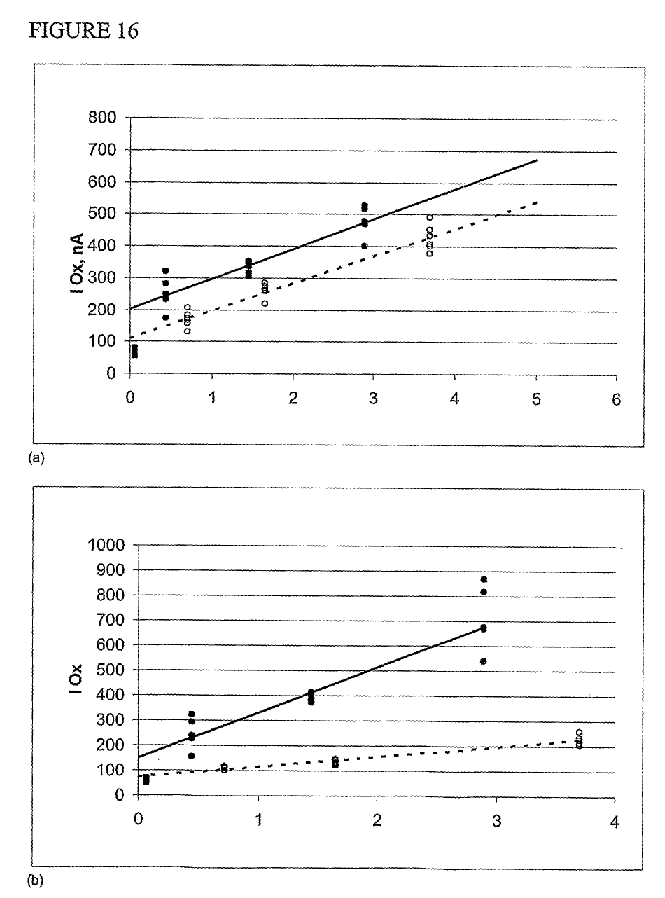

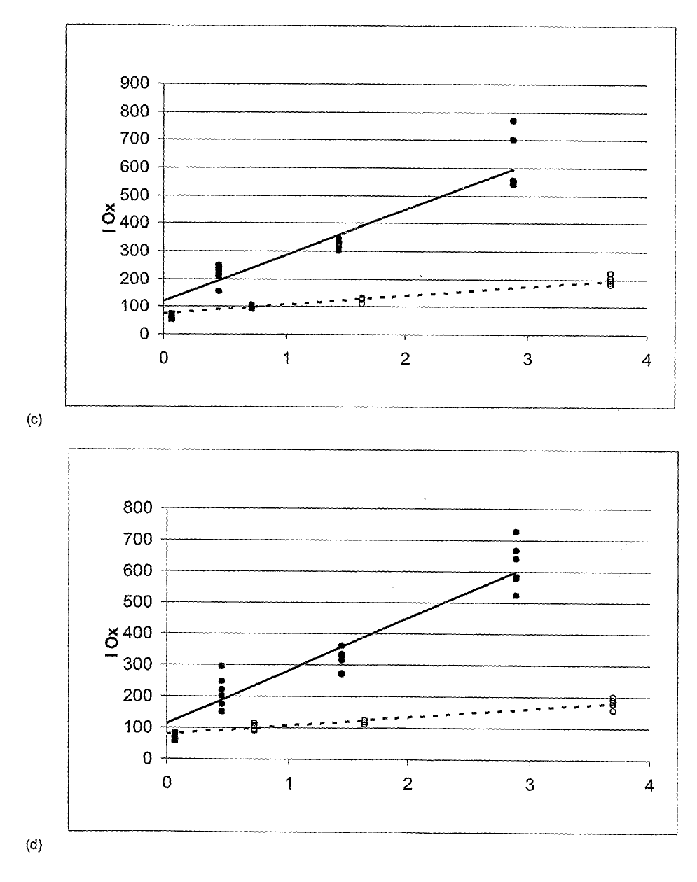

Results are depicted in FIGS. 15 and 16 and Table 14.

TABLE 14
Differentiation with different concentrations of SMC at 98 seconds

Surfactant
concentration

| | 0% | 5% | 7.5% | 10% |
|---|---|---|---|---|
| | SMC | SMC | SMC | SMC |
| HDL Gradient | 94.4 | 182.2 | 164.2 | 169.4 |
| (mM/nA) | | | | |
| LDL Gradient | 86.0 | 40.9 | 32.5 | 27.3 |
| (mM/nA) | | | | |
| Differentiation (%) | 8.9 | 77.5 | 80.2 | 83.9 |

### Example 21

### Solution Preparation, Dispense and Freeze Drying

Several solutions were prepared using the same basic enzyme mix but different surfactants such that the final concentrations of components were:

### 0.1M Tris-HCL, pH 9.0

50 mg/ml (5%) Glycine
Surfactant (0% SMD or no surfactant (0 mg/ml), 0.5% Sucrose Monododecanoate (SMD) (5 mg/ml), 1.0% SMD (10 mg/ml) or 1.5% (15 mg/ml))

### 80 mM Ruthenium Hexaamine Chloride

6 mg/ml Thio-nicotinamide adenine dinucleotide (TNAD)
4 mg/ml Putidaredoxin Reductase (PdR)
3 mg/ml Genzyme lipase
22.2 mg/ml Cholesterol Dehydrogenase

0.4 ul of solution was dispensed per well onto sensors as described in WO 200356319. The sensors were freeze dried.

### Testing Procedure

20 ul of Scipac HDL or LDL prepared in delipidated serum was added to each electrode. The electrodes were tested by chronoamperometry using an Autolab and a multiplexer. At t=0 seconds the chronoamperometry tested was initiated. The oxidation current was measured at +0.15V for 1 second at 14 second consecutive time intervals, for a period of 140 seconds, followed by a reduction current at −0.45V for 1 second. There was a 14 second delay between oxidations which resulted in oxidations at approximately 0, 14, 28, 42, 56, 70, 84, 98, 112, 126 and 140 seconds. Data was analysed for current values at 1 second on the transient.

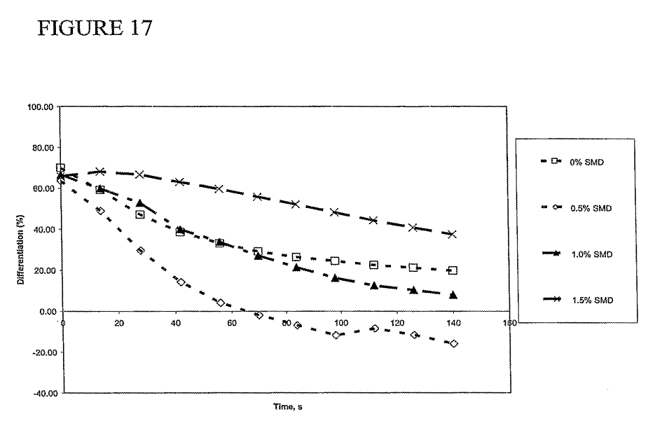

Results are depicted in FIGS. 17 and 18 and Table 15.

TABLE 15
Differentiation with different concentrations of SMD at 98 seconds

Surfactant
concentration

| | 0% | 0.5% | 1% | 1.5% |
|---|---|---|---|---|
| | SMD | SMD | SMD | SMD |
| HDL Gradient | 103.6 | 70.8 | 93.5 | 110.5 |
| (mM/nA) | | | | |
| LDL Gradient | 78.1 | 79.1 | 78.2 | 57.7 |
| (mM/nA) | | | | |
| Differentiation (%) | 24.5 | −11.8 | 16.32 | 48.29 |

1. A method for the determination of the amount of cholesterol in high density lipoproteins in a high density lipoprotein containing sample, said method comprising reacting the sample with (a) a surfactant which selectively breaks down high density lipoproteins and which optionally attenuates the reaction of low density lipoproteins, said surfactant being selected from sucrose esters and maltosides, and measuring the amount of cholesterol in the high density lipoproteins.

2. A method according to claim 1, wherein the surfactant (a) has a differentiation for HDL over LDL given by the equation (i) below of at least 50%:
$$\begin{matrix}Differentiation ( \% ) = \frac{{G}_{HDL} - {G}_{LDL}}{{G}_{HDL}} \times 100 \\ wherein {G}_{HDL} is \frac{measured ( HDLcholesterol )}{known [ HDLcholesterol ]} , \\ and {G}_{LDL} is \frac{measured ( LDLcholesterol )}{known [ LDLcholesterol ]} .\end{matrix} \qquad ( i )$$
wherein measured (HDL cholesterol) or measured (LDL cholesterol) is a measured value which relates to the HDL or LDL cholesterol concentration respectively.

3. A method according to claim 1, wherein the surfactant is (i) a sucrose ester comprising a sucrose moiety in which one or more HO— groups is independently replaced with a group RCOO—, wherein R is an alkyl or alkenyl group having up to 18 carbon atoms, or (ii) a β-maltoside of formula (II) 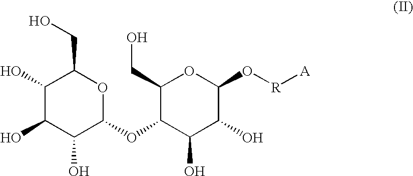
wherein R is an alkylene or alkenylene group having up to 18 carbon atoms and A is methyl or a cycloalkyl group having from 4 to 7 carbon atoms, or a corresponding α-maltoside.

4. A method according to claim 1 to 3, wherein the surfactant is sucrose monocaprate, cyclohexylbutyl-β-D-maltoside (Cymal-4) or cyclohexylpentyl-β-D-maltoside (Cymal-5).

5. A method according to claim 1, wherein the amount of cholesterol in the high density lipoproteins is measured by reacting the sample with (b) a cholesterol ester hydrolysing reagent and (c) cholesterol oxidase or cholesterol dehydrogenase and determining the amount of cholesterol which has reacted with the cholesterol oxidase or cholesterol dehydrogenase.

6. A method according to claim 1, wherein the amount of cholesterol in the high density lipoproteins is measured by an electrochemical technique.

7. A method according to claim 6, wherein the method comprises reacting the sample with
(a) a surfactant which selectively breaks down high density lipoproteins and which optionally attenuates the reaction of low density lipoproteins;
(b) a cholesterol ester hydrolysing reagent;
(c) cholesterol oxidase or cholesterol dehydrogenase;
(d) a coenzyme; and
(e) a redox agent capable of being oxidised or reduced to form a product;
and electrochemically detecting the amount of product formed.

8. A method according to claim 7, wherein the cholesterol ester hydrolysing reagent is a lipase.

9. A method according to claim 7, wherein the sample is additionally reacted with (f) a reductase.

10. A method according to claim 7, wherein the sample is reacted simultaneously with the surfactant (a), the cholesterol ester hydrolysing reagent (b) and the cholesterol oxidase or cholesterol dehydrogenase (c).

11. A method according to claim 1, wherein the high density lipoprotein containing sample is whole blood and wherein the method additionally comprises the step of filtering the sample to remove red blood cells.

12. A method according to claim 1, wherein the measurement of the amount of cholesterol in the high density lipoproteins is completed within a period of no more than 3 minutes from reaction of the sample with the surfactant.

13. A reagent mixture for use in a method for the determination of the amount of cholesterol in high density lipoproteins in a high density lipoprotein containing sample, the reagent mixture comprising
(a) a surfactant which selectively breaks down high density lipoproteins and which optionally attenuates the reaction of low density lipoproteins said surfactant being selected from sucrose esters and maltosides;
(b) a cholesterol ester hydrolysing reagent; and
(c) cholesterol oxidase or cholesterol dehydrogenase.

14. A reagent mixture according to claim 13, which additionally comprises (d) a coenzyme, (e) a redox agent capable of being oxidised or reduced to form a product; and optionally (f) a reductase.

15. A kit for the determination of the amount of cholesterol in high density lipoproteins in a high density lipoprotein containing sample, the kit comprising (a) a surfactant which selectively breaks down high density lipoproteins and which optionally attenuates the reaction of low density lipoproteins, said surfactant being selected from sucrose esters and maltosides, (b) a cholesterol ester hydrolysing reagent, and (c) cholesterol oxidase or cholesterol dehydrogenase, and optionally one or more of (d) a coenzyme, (e) a redox agent capable of being oxidised or reduced to form a product, and (f) a reductase, and means for measuring the amount of cholesterol which reacts with the cholesterol oxidase or cholesterol dehydrogenase.

16. A kit according to claim 15 wherein the means for measuring the amount of cholesterol which reacts with the cholesterol oxidase or cholesterol dehydrogenase comprises
an electrochemical cell having a working electrode, a reference or pseudo reference electrode and optionally a separate counter electrode;
a power supply for applying a potential across the cell; and
a measuring instrument for measuring the resulting electrochemical response.

17. A kit according to claim 15, wherein the reagents (a), (b) and (c) and optionally one or more of (d), (e) and (f) are present in the form of a single reagent mixture.

18. A kit according to claim 17, wherein the reagent mixture is in dried form.

19. A method of operating a kit for the determination of the amount of cholesterol in high density lipoproteins in a high density lipoprotein containing sample, the kit comprising (a) a surfactant which selectively breaks down high density lipoproteins and which optionally attenuates the reaction of low density lipoproteins said surfactant being selected from sucrose esters and maltosides, (b) a cholesterol ester hydrolysing reagent, and (c) cholesterol oxidase or cholesterol dehydrogenase, and optionally one or more of (d) a coenzyme, (e) a redox agent capable of being oxidised or reduced to form a product, and (f) a reductase, and means for measuring the amount of cholesterol which reacts with the cholesterol oxidase or cholesterol dehydrogenase, said method comprising
(i) contacting (1) the reagents (a), (b) and (c), and optionally one or more of (d), (e) and (f), and (2) a high density lipoprotein containing sample, with each other and with the electrodes;
(ii) applying a potential across the electrochemical cell; and
(iii) electrochemically detecting the amount of product formed by measuring the resulting electrochemical response.

20. A method according to claim 19, wherein step (iii) is completed within a period of up to 3 minutes from the time at which the sample is contacted with the reagents (a), (b) and (c).
